(19) United States
(12) Patent Application Publication
(10) Pub. No.: US 2018/0280556 A1
(43) Pub. Date: Oct. 4, 2018

(54) PREDICTIVE ANALYTICS FOR CREATING SENSORY STIMULI EVENTS

(71) Applicant: Eye Labs, LLC, Mountain View, CA (US)

(72) Inventor: Sina FATEH, Mountain View, CA (US)

(21) Appl. No.: 15/491,721

(22) Filed: Apr. 19, 2017

Related U.S. Application Data
(60) Provisional application No. 62/480,223, filed on Mar. 31, 2017.

(51) Int. Cl. A61L 9/12; G06K 9/00; G02B 27/01

(52) U.S. Cl. CPC A61L 9/12; G06K 9/00671; G02B 27/0093; G02B 27/01; G02B 27/017

(57) ABSTRACT

Introduced herein are techniques for bringing scented advertisements into digital content (e.g., visual content and non-visual content). Such technology is able to address a fundamental challenge that is inherent in digital transactions, namely, an inability to accurately gauge different characteristics of a product. Several embodiments pertain to scent delivery systems that are able to produce scent(s) as part of a direct advertising campaign or an indirect advertising campaign. More specifically, a scent delivery system may be configured to produce scents that correspond to features of the digital content. For example, different scented materials can be dispensed based on characteristics of a product that is the subject of a scented advertisement.

### CROSS-REFERENCE TO RELATED APPLICATIONS

This application claims priority to U.S. Provisional Patent Application No. 62/480,223 titled “SCENT MARKETING AND ADVERTISING FOR HEAD-MOUNTED DISPLAYS” and filed on Mar. 31, 2017, which is incorporated by reference herein in its entirety.

### RELATED FIELD

The present technology relates to the production of sensory stimuli and, more specifically, accessories for electronic devices that produce sensory stimuli to increase the realism of content presented by the electronic devices.

### BACKGROUND

Smell is one of our strongest senses. Certain scents often remind us of moments (both good and bad) that we first experienced days, weeks, or years ago. Scents can also make be highly effective in helping us distinguish items or locations from one another.

Some businesses (e.g., production studios, movie theaters, and amusement parks) have long attempted to enhance interactive experiences by introducing scents. For example, “Smell-O-Vision” was a system that released scents during the projection of a film so that viewers could “smell” what was happening in the movie. Similarly, the “Smellitizer” was developed by the Walt Disney Company to deliver faint scents on cue at certain locations, such as attractions (e.g., Soarin' Over California® at Disneyland®) and storefronts (e.g., Main Street Bakery). Scent dispersal systems such as these were often shown to heighten the realism of a fabricated experience, increase sales, and improve customer satisfaction.

Accordingly, entities across different industries have begun using scent as part of a multi-sensory marketing strategy designed to enhance an individual's experience (e.g., of a location or a particular product/service). These enhanced experiences allow customers to develop deeper memories and emotional connections with certain brands, products, services, locations, etc. In fact, some brands have begun using scent to distinguish themselves in the market. Such techniques are generally performed as part of a scent marketing campaign.

Scent marketing campaigns can typically be readily introduced by merchants who occupy a physical storefront. However, scent marketing becomes increasingly difficult as merchants complete more transactions electronically and reduce the number of physical storefronts. Said another way, the proliferation of electronic transactions has made scent marketing more difficult because scents must be delivered to prospective customers.

### SUMMARY

Smell is a powerful sense that has the ability to evoke different feelings and memories, as well as impact a consumer's experience during a transaction. In fact, smell may influence whether the consumer ultimately decides to purchase certain types of products, such as cosmetics, perfumes, colognes, soaps, shampoos, personal hygiene items, etc.

For example, fragrance strips have been placed in magazines to market such products since the 1960s. Studies have shown that fragrance strips are very effective in grabbing a reader's attention. On average, 64% of magazine readers try at least one fragrance strip (or another form of scent-based advertisement) if fragrance strip(s) are placed in a magazine. Studies have also shown that these scented advertisements increase product sales by attracting more attention than unscented advertisements.

Introduced here, therefore, are systems and techniques for bringing scented advertisements into other forms of content, including visual content (e.g., augmented and virtual reality content) and non-visual content (e.g., audiobooks). Such technology can be used to address a fundamental challenge that is inherent in digital transactions (i.e., an inability to accurately gauge different characteristics of a product) by engaging a consumer's sense of sight (vision), hearing (audition), taste (gustation), smell (olfaction), and/or touch (somatosensation). Stimuli may also be produced for other sensory modalities such as temperature (thermoception).

More specifically, a stimuli delivery system may produce a sensory stimulus (e.g., a scent) that is related to the content being experienced by an individual. For example, the stimuli delivery system (also referred to as a “scent delivery system”) may produce a scent that is related to a product shown within a virtual or augmented reality environment. The product may be the subject of a direct advertisement or an indirect advertisement (e.g., as part of a cinematic film or video game). The term “product” includes goods and/or services that are available for purchase by the individual.

If the individual indicates an interest in the product, the individual may be asked whether she wishes to purchase the product. This can occur in several different ways. In some embodiments, a notification may be presented by the computing device used to view the visual content (e.g., on a head-mounted display (HMD) or a network-connected television). The notification may prompt the individual to approve a purchase of the product, input financial information (e.g., name, address, and payment card details), decline a purchase of the product, or add the product to a queue (often referred to as a “shopping cart”) for further review. In other embodiments, a notification may be presented by another computing device that is communicatively coupled to the computing device used to view the visual content. For example, the individual may receive notifications on a mobile phone while viewing visual content on an HMD or a network-connected television.

Also introduced here are techniques for generating emotional input based on characteristics of an individual (referred to as “user characteristics”). For example, an emotional input may be generated based on the pulse rate or temperature of an individual. These features could be measured by an internal sensor that is integrated into the computing device (e.g., an HMD) used to view visual content or an external sensor that is integrated into some other computing device (e.g., a fitness tracker or Microsoft Kinect®). Emotional input may affect the sensory stimuli that are produced by the scent delivery system. For example, scents may be produced less frequently or with less intensity as an individual's heart rate and/or temperature begin to rise.

The individual may also have the option of placing the scent delivery system in automatic mode or manual mode. In automatic mode, the scent delivery system decides which scent(s) should be produced and when those scent(s) should be produced. In manual mode, the individual can either turn off the scent functionality entirely or personalize the delivery parameters (e.g., by specifying a delivery time, a delivery duration, a scent intensity, which type(s) of content should have scent functionality, which scent(s) should be produced, which scent(s) should not be produced, etc.).

### BRIEF DESCRIPTION OF THE DRAWINGS

Embodiments of the present invention are illustrated by way of example and not limitation in the accompanying drawings, in which like references indicate similar elements. Various objects, features, and characteristics of the present invention will become more apparent to those skilled in the art from a study of the Detailed Description in conjunction with the accompanying drawings.

FIG. 1A depicts a conventional head-mounted display (HMD) that includes an image display system and an optical display surface configured to reflect light.

FIG. 1B depicts a conventional HMD that includes an image display system and an optical display surface configured to reflect light.

FIG. 2A is an inside view of a binocular HMD that includes a scent delivery system.

FIG. 2B is a front view of the binocular HMD that includes a scent delivery system.

FIG. 3 depicts an example of a network environment that includes an HMD having a scent delivery system, a content server, and another computing device.

FIG. 4 depicts a scent delivery system that can be affixed to an HMD configured to display augmented or virtual reality content.

FIG. 5 depicts a development platform that allows visual content to be specially designed for presentation by a computing device that is connected to a scent delivery system.

FIG. 6A depicts an example of a video track that includes a keyframe identified through image segmentation.

FIG. 6B depicts an example of an audio track that includes a keyframe identified through audio segmentation.

FIG. 7 depicts an interface through which a user can browse items offered for sale by an electronic commerce platform.

FIG. 8A depicts a digital kitchen environment that is viewable through an HMD.

FIG. 8B depicts a digital garden environment that is viewable through an HMD.

FIG. 9 includes an overhead map illustrating how a digital environment may include one or more scented regions.

FIG. 10A depicts an order interface that may be presented by a computing device (e.g., an HMD or a network-connected television) that is used to view visual content.

FIG. 10B depicts an order interface that may be presented by another computing device (e.g., a mobile phone or a wearable device, such as a fitness tracker or watch) that is communicatively coupled to a computing device used to view visual content.

FIG. 11 depicts a process for generating an instruction set for a scent delivery system.

FIG. 12 depicts a process for controllably producing scents that correspond with digital content experienced by a user.

FIG. 13 is a block diagram illustration an example of a processing system in which at least some operations described herein can be implemented, consistent with various embodiments.

The figures depict various embodiments for the purpose of illustration only. One skilled in the art will readily recognize that alternative embodiments of the technology described herein may be employed without departing from the principles of the present invention.

### DETAILED DESCRIPTION

Introduced here are computer-implemented techniques and systems for increasing the effectiveness of marketing, advertising, and branding campaigns that target consumers of digital content by controllably producing certain sensory stimuli. Stimuli delivery systems (also referred to as “scent delivery systems”) are described herein that can produce different sensory stimuli based on the digital content being consumed at a given point in time. For example, a scent delivery system may produce one or more scents based on visual content being shown on a head-mounted display (HMD). A scent delivery system may be separately attachable to a computing device used to view the visual content or may be integrated within the computing device itself.

Although many of the embodiments described herein involve head-mounted displays (HMDs), one skilled in the art will recognize that such embodiments have been selected for the purpose of illustration only. Such technology could be used in combination with any computing device that presents content, including personal computers, tablet computers, personal digital assistants (PDAs), mobile phones, game consoles (e.g., Sony PlayStation® or Microsoft Xbox®), music players (e.g., Apple iPod Touch®), wearable electronic devices (e.g., a watch or fitness band), network-connected (“smart”) devices (e.g., a television), virtual/augmented reality systems (e.g., Oculus Rift® or Microsoft Hololens®), or other electronic devices.

For example, an individual could wear a network-connected mask (or some other form of headwear) that includes a scent delivery system but allows the individual to view visual content shown by another computing device (e.g., a network-connected television). Accordingly, the network-connected mask may be communicatively coupled to one or more other computing devices. The network-connected mask and the other computing device(s) may communicate with one another via a wired connection or a wireless connection. In some embodiments, the network-connected mask and the other computing device(s) communicate via a short-range wireless technology/protocol, such as Wi-Fi, Bluetooth, near-field communication (NFC), cellular radio, infrared, radio-frequency identification (RFID), etc.

A scent delivery system can include one or more reservoirs of scented material (e.g., liquid or air) that can each be selectively dispensed by a pump at the appropriate time. That is, the scent delivery system includes pump(s) for controllably dispensing the scented material(s) in certain quantities/mixtures at certain times. For example, a pine scent could be produced when the individual is able to view a digital forest, while a vanilla scent could be produced when the individual is able to view a digital kitchen. In some embodiments, the scented material(s) are housed within capsules that are removable from the scent delivery system and readily replaceable. Accordingly, the reservoir may be refillable only when the attachment is separated from the head-mounted device or modularly replaceable while the scent delivery system is connected to the head-mounted device.

Unlike traditional scent dispersal systems (e.g., the Smell-O-Vision or the Smellitizer), the scent delivery systems described herein are portable and allow scents to be delivered directly to a single user of an HMD. Said another way, traditional scent dispersal systems are designed to dispense scents in a single physical location, while the scent delivery systems introduced here can be used regardless of where the user decides to experience visual content.

Consequently, scent delivery systems can be used to increase the effectiveness of marketing, advertising, and branding campaigns that target consumers of digital content by controllably producing certain scent(s). Several different use cases illustrate the benefits of enhancing the realism of digital content (e.g., visual content and non-visual content) by replacing unrelated real-world stimuli with controlled stimuli.
Direct Advertising: A scent delivery system can produce an appropriate scent while an individual watches an advertisement for an item (e.g., a perfume or a deodorant). The individual may also be permitted to buy the item in real time during the advertisement. For example, if the individual exhibits an interest in the scent (e.g., by moving her head closer to the digital source of the scent), the individual may be prompted to decide whether she would like to order the item directly through the advertisement. That is, the individual may be able to complete a digital transaction without disrupting the current experience by navigating to a separate channel.
Indirect Advertising: Scent(s) related to digital content experienced by an individual can be intelligently replicated by a scent delivery system, which allows products already present in the digital content to be indirectly advertised to consumers. For example, the scent delivery system may produce a perfume scent when a female character in a cinematic film approaches the camera. The individual may be able to order the item in real time (i.e., as the digital content continues to run) and/or save a product summary for subsequent ordering. Moreover, a list of product(s) indirectly advertised throughout the runtime of the digital content could be shown to the individual following the conclusion of the digital content. In such instances, the individual may prompt the scent delivery system to reproduce scent(s) that were previously produced during the runtime of the digital content. Indirect advertising techniques are particularly useful for those forms of content where direct advertising is undesirable (e.g., due to content flow disruptions) or difficult to carry out (e.g., due to the presence of ad blockers).
Business Model Improvements: Merchants (i.e., manufacturers/sellers of cosmetics, perfumes, food, beverages, etc.) can work with fragrance companies (i.e., manufacturers of scented materials) and development companies (e.g., manufacturers of scent delivery systems and producers of digital content) to re-create scents that will improve the effectiveness of advertising campaigns, the desirability of products, etc. For example, some or all of these entities may work together to develop a scented advertisement and generate instructions for producing the appropriate scent(s) at the appropriate time(s).
Promotional Cartridges: Merchants could send promotional capsules that include scented materials out on a periodic (e.g., weekly or monthly) basis. For example, a perfume manufacturer may distribute promotional capsules each time a new perfume is developed. As another example, the perfume manufacturer may distribute promotional capsules as part of the concept testing process that typically occurs prior to production (e.g., multiple capsules could be delivered to potential consumers who are asked to identify their favorite scent or rate the various scents).

Accordingly, visual content may be specially designed for presentation by a computing device that is connected to a scent delivery system. In such embodiments, a processor can determine whether to dispense a scented material based on instructions that are automatically generated based on characteristics of the corresponding visual content or manually generated by an administrator. The administrator may be associated with a merchant, a fragrance company, or a development company. The administrator may generate the instructions by working with one or more of these entities to identify the combination of one or more scented materials needed to accurately reproduce a certain scent. The instructions could also be automatically generated based on, for example, visually identifiable elements in individual frames, audio events, etc.

The instructions may also specify a delivery timestamp, an intensity/quantity of each scented material, etc. In some embodiments the instructions are embedded within the digital content (e.g., encoded in a video track, audio track, and/or metadata), while in other embodiments the instructions are included in a distinct instruction set that accompanies the digital content and is separately executable by the scent delivery system. Similar techniques could be used for visual content (e.g., applications, games, and cinematic videos) and non-visual content (e.g., audiobooks, concerts, and radio programs).

Older, unscented digital content could also be repurposed for use with a computing device having a scent delivery system. An administrator may be able to tag unscented digital content to create keyframes that cause the scent delivery system to produce scent(s). For example, the administrator may identify keyframes in a direct advertisement that will benefit from the production of a scent. As another example, the administrator may identify keyframes in existing digital content (e.g., a cinematic film) in order to create an indirect advertisement.

Content modification may be completed using a graphical user interface (GUI) that is supported by a repurposing engine. More specifically, the administrator could upload unscented digital content to the GUI, which prompts the repurposing engine to analyze the unscented digital content to determine whether scent(s) should be produced and, if so, which scent(s) should be produced. For example, in some embodiments the repurposing engine automatically identifies candidate instances for dispensing scented material(s). The candidate instances typically include one or more features (e.g., a character, a weather event, or a product, such as a perfume bottle or a piece of fruit) that are readily recognizable upon performing content analysis. Content analysis may require the repurposing engine execute image processing algorithms, audio processing algorithms, etc. As another example, in some embodiments the repurposing engine automatically determines characteristics regarding the production of each scent. The characteristics could include how much scented material should be dispensed (e.g., whether the aroma should be faint, medium, or strong), when the scented material should be dispensed, whether a combination of multiple scented materials is necessary to produce a certain scent, etc. The administrator may be able to modify the instances and/or characteristics proposed by the repurposing engine. The administrator may also be able to specify additional instances and/or characteristics using the GUI.

Disjointed experiences will often occur if scents are produced too early or too late. Accordingly, predictive analytics may be employed to determine when, exactly, a scent should be produced for a given keyframe. Such techniques may prompt a scent delivery system to begin producing the scent before the keyframe is shown or heard. For example, the lead time could be measured by duration (e.g., 50 milliseconds) or frame count (e.g., 5 frames).

Thus, unscented digital content could be manually repurposed into scented digital content by an administrator and/or automatically repurposed into scented digital content by the repurposing engine. The repurposing engine may also engage in machine learning to identify candidate instances and keyframes, identify the appropriate scent(s) to be produced at the keyframes, etc. For example, the repurposing engine may monitor the overall effectiveness of a scent marketing campaign by analyzing different quantitative metrics, such as the merchant's retail traffic, click-through rates, conversion rates, etc. Such techniques allow the repurposing engine to identify the characteristics (e.g., scent type, scene type, and content type) that correspond with effective scent marketing campaigns, and then use this information to more intelligently produce scents that align with content. For example, the repurposing engine may discover that scented advertisements for a perfume product are much more effective when shown after romance films rather than action films or comedy films.

Various machine learning algorithms and techniques could be employed by the repurposing engine, including Naïve Bayes Classifier algorithms, K Means Clustering algorithms, Support Vector Machine algorithms, linear regression, logic regression, artificial neural networks, etc. These machine learning algorithms/techniques may be chosen based on application (e.g., supervised or unsupervised learning) and optimized based on whether the administrator has confirmed candidate instances proposed by the repurposing engine, indicated that a certain scent created a more realistic or desirable experience, indicated that a certain scent led to more effective advertising campaigns (e.g., measured by increased viewer engagement or increased product sales), etc.

Note that the tasks performed by the repurposing engine could also be performed by a series of dedicated modules. For example, a processing module may be responsible for identifying candidate instances, a stimuli creation module may be responsible for identifying the scented material(s) needed to recreate a certain scent, and an analytics module may be responsible for monitoring the effectiveness of a scent marketing campaign.

### Terminology

Brief definitions of terms, abbreviations, and phrases used throughout the specification are given below.

Reference in this specification to “one embodiment” or “an embodiment” means that a particular feature, structure, or characteristic described in connection with the embodiment is included in at least one embodiment of the disclosure. The appearances of the phrase “in some embodiments” are not necessarily referring to the same embodiments, nor are they necessarily referring to separate or alternative embodiments that are mutually exclusive of one another. Moreover, various features are described that may be exhibited by some embodiments but not others. Similarly, various requirements are described that may be requirements for some embodiments but not others.

Unless the context clearly requires otherwise, the words “comprise” and “comprising” are to be construed in an inclusive sense rather than an exclusive or exhaustive sense (i.e., in the sense of “including, but not limited to”). The terms “connected,” “coupled,” or any variant thereof includes any connection or coupling, either direct or indirect, between two or more elements. The coupling or connection between the elements can be physical, logical, or a combination thereof. For example, two devices may be coupled directly to one another or via one or more intermediary channels/devices. Devices may also be coupled in such a way that information can be passed there between, despite not sharing any physical connection with one another. The words “associate with,” meanwhile, mean connecting or relating objects, items, etc.

Where the context permits, words used in the singular sense or the plural sense may also be used in the plural sense or the singular sense, respectively. The word “or,” in reference to a list of two or more items, covers all of the following interpretations of the word: any of the items in the list, all of the items in the list, and any combination of the items in the list. If the specification states a component or feature “may,” “can,” “could,” or “might” be included or have a characteristic, that particular component or feature is not required to be included or have the characteristic in every embodiment.

The term “module” refers broadly to software, hardware, and/or firmware components. Modules are typically functional components that can generate useful data or other output using specified input(s). A module may or may not be self-contained. A software program or application may include one or more modules.

The terminology used in the Detailed Description is intended to be interpreted in its broadest reasonable manner, even though it is being used in conjunction with certain embodiments. The terms used in this specification generally have their ordinary meanings in the art, in the context of the disclosure as a whole and in the specific context where each term is used. For convenience, certain terms may be highlighted using, for example, capitalization, italics, and/or quotation marks. However, the use of highlighting has no influence on the scope and meaning of a term. The scope and meaning of a term is the same, in the same context, whether or not it is highlighted.

Consequently, although alternative language and synonyms may be used for some terms, special significance is not to be placed upon whether or not a term is elaborated or discussed herein. A recital of one or more synonyms does not exclude the use of other synonyms. The use of examples anywhere in this specification, including examples of any terms discussed herein, is intended to be illustrative only. These examples are not intended to limit the scope and meaning of the disclosure or of any exemplified term. Likewise, the disclosure is not limited to various embodiments described below.

### System Overview

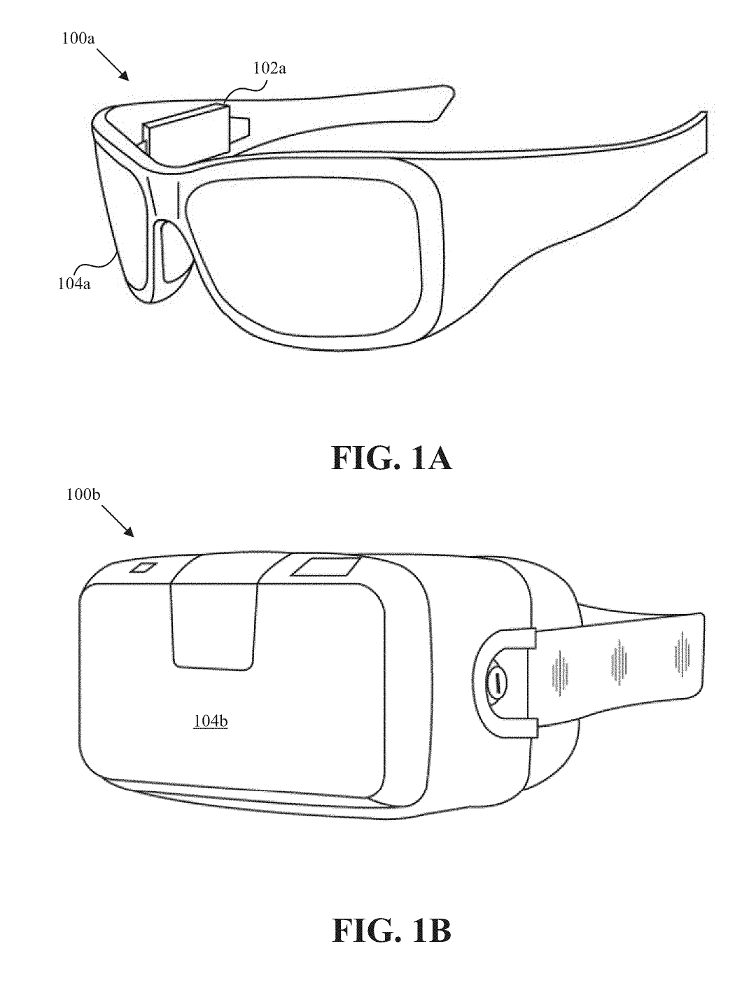

FIGS. 1A-B depict conventional head-mounted displays (HMDs) 100a-b that each include an image display system 102a and an optical display surface 104a-b configured to reflect or emit light. Some conventional HMDs are designed so that visual content emitted by the image display system 102a is reflected by the optical display surface 104a-b towards the user's eyes. Other conventional HMDs are designed so that visual content created/processed by the image display system 102a is projected directly into the user's eyes by the optical display surface 104a-b.

Conventional HMDs 100a-b can cover one or both of the user's eyes. More specifically, binocular HMDs present a separate image to each of the user's eyes, while bi-ocular HMDs present a single image to both of the user's eyes, and monocular HMDs present a single image to only one of the user's eyes. However, as shown in FIGS. 1A-B, conventional HMDs 100a-b do not cover the user's nose or mouth, which separates or isolates several critical senses. For example, the HMDs 100a-b shown here isolate the olfactory sense and the gustatory sense from the visual sense.

Consequently, a user of a conventional HMD will continue to receive stimuli from the real world that can disrupt the realism of the augmented or virtual reality content shown by the HMD. In fact, some of these stimuli may be partially or entirely counter to what the user expects. For example, the user might smell a fried chicken scent that is emanating from the kitchen while viewing a dissimilar virtual environment (e.g., the inside of an spaceship) through an HMD. Unrelated stimuli are likely to cause the user to have a disjointed and unrealistic experience while using the HMD.

HMDs can be configured to display two different types of digital imagery. First, some HMDs are configured to display simulated (e.g., computer-generated) digital images that together form an entirely virtual environment. This is referred to as “virtual reality.” Virtual reality generally requires the user wear a helmet, goggles, or some other headwear that forms an enclosed area around the user's eyes, thereby blocking out the real world. Second, some HMDs are configured to display simulated (e.g., computer-generated) images that are superimposed onto real-world content perceived by the user. This is referred to as “augmented reality.” Augmented reality allows the user to concurrently view virtual imagery and real-world imagery and, in some instances, interact with virtual content that relates to what is seen the real world. Generally, the user is able to distinguish between the virtual content and the real-world imagery. For example, augmentation may take place through superposition of a simulated image on a non-simulated, real-world environment. The simulated image may allow the user to view additional data relevant to a desired task, the non-simulated, real-world environment, etc. The simulated image could also be adapted to allow for user interaction. However, augmented and virtual reality experiences are generally limited in how realistic they are because they are largely visual experiences.

HMD systems fall into three general categories: binocular systems, bi-ocular systems, and monocular systems. Binocular HMD systems present a separate image to each of the user's eyes, while bi-ocular HMD systems present a single image to both of the user's eyes, and monocular HMD systems present a single image to only one of the user's eyes. One skilled in the art will recognize that the systems and techniques described herein can be adapted for any of these systems. The use of one or more of these terms in describing an embodiment is for the purpose of illustration only.

HMDs enable users to more easily interact with augmented or virtual reality assets. The assets can include, for example, network-accessible interfaces (e.g., that are accessible through a web browser), software programs/applications, video games, and cinematic videos. HMDs could also be designed to facilitate non-visual experiences. For example, many conventional HMDs include (or can be easily paired with) headphones or speakers through which audio assets (e.g., audiobooks, concerts, and radio programs) can be projected.

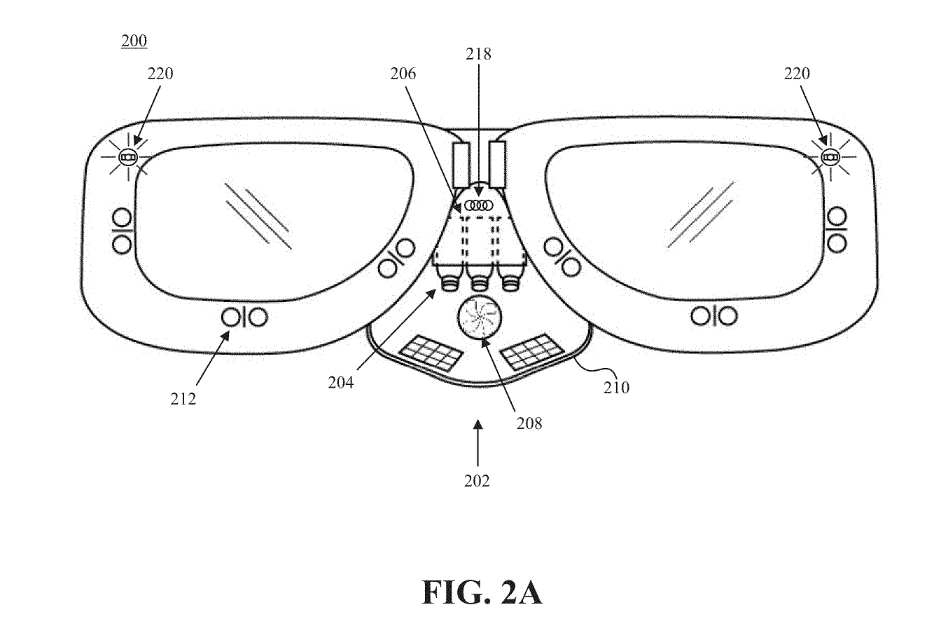

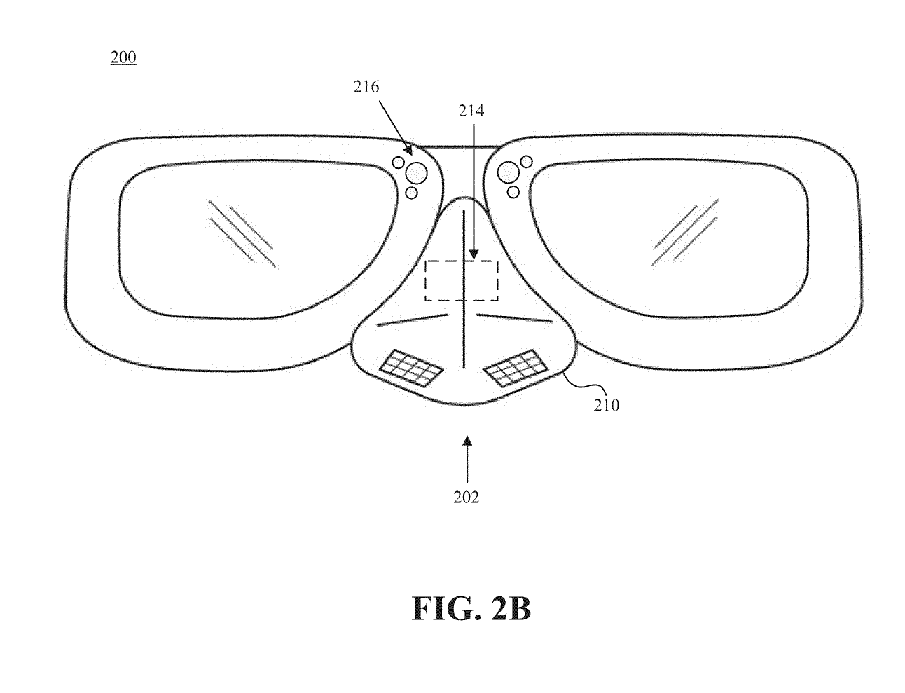

FIG. 2A is an inside view of a binocular HMD 200 that includes a scent delivery system 202. FIG. 2B is a front view of the binocular HMD 200 that includes a scent delivery system 202. The scent delivery systems described herein enhance the realism of digital content (e.g., visual content and non-visual content) experienced by a user by replacing real-world stimuli with controlled stimuli that are related to the digital content. For example, the scent delivery systems can increase the realism of digital content by producing scent(s) that trick the user's olfactory sense (i.e., sense of smell).

A scent delivery system 202 can be completely self-contained, completely integrated into the binocular HMD 200, or partially self-contained and partially integrated into the binocular HMD 200. In some embodiments, the scent delivery system 202 is detachably connectable to the frame of the binocular HMD 200 using one or more fasteners (e.g., magnets, mechanical clips/tracks, or some other quick release mechanism). The fastener(s) allow the scent delivery system 202 to be readily attached to, and then removed from, the binocular HMD 200 without the use of tools. For example, the structural frame of the binocular HMD 200 may include features (e.g., cavities) configured to mate with corresponding features (e.g., protrusions) of a scent delivery system 202. In other embodiments, the scent delivery system 202 is fixedly attached to the frame of the binocular HMD 200 using, for example, screws, nuts and bolts, or an adhesive material/substance.

Additionally or alternatively, some or all of the components that enable scent to be dispensed (e.g., pumps, reservoirs, and/or fans) could be integrated within the HMD. For example, the pump(s) and reservoir(s) may be integrated into the structural frame of an HMD (which renders a separate attachment unnecessary). In such embodiments, the pump(s) may be arranged such that scented material(s) are ejected toward the user's nose.

One skilled in the art will recognize that embodiments involving a binocular HMD are described herein for the purpose of illustration only. The scent delivery systems and computer-implemented techniques described herein are equally applicable to monocular HMDs, bi-ocular HMDs, and other computing devices (e.g., mobile phones, televisions, and wearable devices). The scent delivery system 202 may be customizable for a particular user, a particular type of experience (e.g., augmented or virtual reality content), a particular HMD (e.g., Oculus Rift® or Microsoft Hololens®), etc.

The scent delivery system 202 can be designed so that it partially or entirely covers the user's nose. For example, FIGS. 2A-B depict a scent delivery system 202 that completely envelops the user's nose. However, the scent delivery system 202 could also be designed so that the base of the user's nose is exposed. In some embodiments, some of the components (e.g., the pumps 204, reservoirs 206, and/or fan 208) are integrated into the structural frame of the binocular HMD 200 so that the binocular HMD 200 appears similar to a conventional HMD (e.g., the HMDs of FIGS. 1A-B) despite supporting scent functionality.

More specifically, the scent delivery system 202 can include a structural frame 210, one or more pumps 204, one or more reservoirs 206, and a fan 208. The structural frame 210 can include fasteners that allow the scent delivery system 202 to be readily connected to, and then removed from, the binocular HMD 200. For example, the structural frame 210 could include features (e.g., detents or cavities) configured to mate with corresponding features (e.g., protrusions) of the scent delivery system 202. Examples of fasteners include magnets, mechanical clips/latches/tracks, and other quick release mechanisms. In other embodiments, the scent delivery system 202 is fixedly attached to the structural frame 210 using screws, nuts and bolts, adhesive materials/substances, etc.

The pump(s) 204 allow a scented material (e.g., liquid or air) stored in each of the reservoir(s) 206 to be dispensed in a controlled manner. For example, the pump(s) 204 could dispense scented liquids in particular quantities, at particular temperatures, with particular forces, etc. In fact, the pump(s) 204 could be collectively controlled by scent circuitry that can intelligently cause dispersal of a single scented material and combinations of multiple scented materials. Said another way, the pump(s) 204 could sequentially or simultaneously dispense the scented material(s) stored in the reservoir(s) 206.

In some embodiments the pump(s) 204 spray a scented material directly toward the user's nose, while in other embodiments the pump(s) 204 spray the scented material within the internal cavity of the structural frame 210 (in which case the fan 208 could direct the scented material towards the user's nose or away from the user's nose). As further described below, the pump(s) 204 may dispense a small quantity of a single scented material when a slight aroma is desired, a large quantity of a single scented material when a strong aroma is desired, small or large quantities of multiple scented materials simultaneously, etc.

Scent could also or instead be delivered via small absorbent pads that are connected to the reservoir(s) 206 and disposed near the user's nose. For example, one or more absorbent pads could be affixed on the interior surface of the structural frame 210 of the scent delivery system 202 (i.e., within the internal cavity). Unlike the pump(s) 204 (which are able to controllably dispense scented materials at certain times), the absorbent pads typically continually dispense small amounts of scented material.

The reservoir(s) 206 generally retain scented material(s) having different scents. For example, the three reservoirs depicted in FIGS. 2A-B may correspond to general scents (e.g., sweet, savory, and floral), specific scents (e.g., cinnamon, citrus, and rose), or some combination thereof. Moreover, the reservoirs 206 may be readily replaceable by the user or some other entity (e.g., the manufacturer of the HMD or a service representative). In such embodiments, the reservoirs could be replaced by “capsules” of scented material that are modularly replaceable, which enables the user to customize which scent(s) can be produced by the scent delivery system 202.

Note, however, that one of the reservoir(s) 206 could include a non-scented material (e.g., water) or a neutrally-scented material that is controllably dispensed within the internal cavity to neutralize other scents. For example, a neutralizing scent (also referred to as a “cleansing scent”) may be created to eliminate strong scents (e.g., a smoke odor or a food-based odor) or unwanted scents (e.g., a skunk odor) the user is unlikely to want to linger. The neutrally-scented material could also include particles (e.g., natural oils) that eliminate scents by attaching to odor molecules to form a neutral compound. Neutrally-scented material(s) are often circulated by the fan 208 to more quickly flush scent(s) from the internal cavity of the structural frame 210. Additionally or alternatively, the scent delivery system 202 could produce a masking scent that overwhelms strong scents or unwanted scents. The masking scent could be, for example, peppermint, spearmint, or anise.

In some embodiments, some or all of the material(s) housed within the reservoir(s) 206 may be functional in nature. One example of a functional material is a scented liquid that includes pheromone(s) that trigger specific response(s), such as alarm pheromones, signal pheromones, sex pheromones, etc. For example, a scent delivery system 202 could dispense a scented or unscented material that includes an alarm pheromone during a horror film or a horror video game.

In some embodiments, the scented material(s) stored in the reservoir(s) 206 also include a flavor compound having a particular taste. Accordingly, the user may experience digital content using her visual, olfactory, and/or gustatory senses. For example, when the user views a digital environment that includes a citrus grove, the scent delivery system 202 may dispense a material having a citrus scent and/or a citrus taste. Scent and taste could be facilitated by a single material or multiple materials. For example, the scent delivery system 202 could include one reservoir that stores a material having a scent-enabling compound and another reservoir that stores a material having a taste-enabling compound. As another example, the scent-enabling compound and the taste-enabling compound may both be included in the same material that is stored in a single reservoir.

Several examples of scent-enabling compounds (also referred to as “aroma compounds”) are provided in Table I, which is not intended to be exhaustive.

TABLE I
Examples of aroma compounds.

| Compound Name | Fragrance | Natural Occurrence |
|---|---|---|
| Geranyl acetate | Fruity, rose | Rose, Floral |
| Methyl formate | Ethereal | — |
| Methyl acetate | Sweet, solvent, | — |
| | nail polish | |
| Methyl propionate | Sweet, fruity | — |
| Methyl propanoate | | |
| Methyl butyrate | Fruity, apple, pineapple | Pineapple |
| Methyl butanoate | | |
| Ethyl acetate | Sweet, solvent | Wine |
| Ethyl butyrate | Fruity, orange, pineapple | — |
| Ethyl butanoate | | |
| Isoamyl acetate | Fruity, banana, pear | Banana plant |
| Pentyl butyrate | Fruity, pear, apricot | — |
| Pentyl butanoate | | |
| Pentyl pentanoate | Fruity, apple | — |
| Octyl acetate | Fruity, orange | — |
| Benzyl acetate | Fruity, strawberry | Strawberry |
| Methyl anthranilate | Fruity, grape | — |
| Myrcene | Woody | Verbena, bay leaf |
| Geraniol | Rose, flowery | Geranium, lemon |
| Nerol | Sweet rose, flowery | Neroli, lemongrass |
| Citral | Lemon | Lemongrass |
| Citronellol | Lemon | Lemongrass, rose, |
| | | pelargonium |
| Linalool | Floral, sweet, woody, | Coriander, sweet basil, |
| | lavender | lavender |
| Nerolidol | Woody, bark | Neroli, ginger, jasmine |
| Limonene | Orange | Orange, lemon |
| Camphor | Camphor | Camphor laurel |
| Menthol | Menthol, peppermint | Mentha (corn mint), |
| | | peppermint |
| Carvone | Caraway, spearmint | Caraway, dill, spearmint |
| Terpineol | Lilac | Lilac, cajuput |
| Alpha-Ionone | Violet, woody | Violet |
| Thujone | Minty | Wormwood, lilac, |
| | | juniper |
| Eucalyptol | Eucalyptus | Eucalyptus |
| Benzaldehyde | Almond | Bitter almond |
| Eugenol | Clove | Clove |
| Cinnamaldehyde | Cinnamon | Cinnamon, cassia |
| Ethyl maltol | Cooked fruit, | — |
| | caramelized sugar | |
| Vanillin | Vanilla | Vanilla |
| Anisole | Anise | Anise |
| Anethole | Anise | Anise, sweet basil |
| Estragole | Tarragon | Tarragon |
| Thymol | Thyme | Thyme |
| Trimethylamine | Fishy, ammonia | — |
| Putrescine | Rotting flesh | Rotting flesh |
| Diaminobutane | | |
| Cadaverine | Rotting flesh | Rotting flesh |
| Pyridine | Fishy | Belladonna |
| Indole | Fecal, flowery | Feces, jasmine |
| Skatole | Fecal | Feces, orange blossoms |
| Furaneol | Strawberry, pineapple, | Strawberry, pineapple, |
| | cotton candy, sweet | buckwheat, tomato |
| Hexanol | Grass, woody | — |
| Anisic aldehyde | Sweet, chocolate, vanilla, | — |
| | strawberry, raspberry, | |
| | apricot | |

The fan 208 could be set to continuously or periodically circulate air around the user's nose. For example, the fan 208 may circulate air when the scent delivery system 202 begins dispensing scented material(s) or when the user enables the fan 202 (e.g., by turning a fan setting to “on”). In some embodiments, the structural frame 210 includes one or more openings (also referred to as “breaks” or “outlets”) that allow air to more easily circulate through the internal cavity of the structural frame 210. These opening(s) may be strategically positioned with respect to the user's noise (e.g., near the nostrils as shown in FIGS. 2A-B) or with respect to the pump(s) 204 and/or fan 208.

The fan 208 may also be used to modify the airflow in or around the internal cavity of the structural frame 210. Different attributes (e.g., acceleration, velocity, and temperature) could be modified to simulate wind that is included in content presented by the binocular HMD 200. For example, the fan 208 may intelligently switch between several fan settings to simulate different airflows while the user views an advertisement for a vehicle. The fan 208 may perform such actions in combination with the pump(s) 204. For example, the fan 208 may generate a high airflow and a pump may dispense a material having a salty flavor (e.g., a salty smell and/or a salty taste) while the binocular HMD 200 presents appropriate content, such as a cinematic video involving the ocean or an advertisement for a golf course near the coast.

In some embodiments, the binocular HMD 200 also includes other structural features or components that enhance the user experience. For example, the binocular HMD 200 may include one or more sensors 212 that are positioned proximate to the user's face. The sensor(s) 212 can measure skin temperature, temperature within the internal cavity, humidity, motion, etc. Scent circuitry 214 may be responsible for modifying which scented material(s) are dispensed and how much of each scented material is dispensed based on signals received from each of the sensor(s) 212. For example, if sensor data indicates that the skin temperature has begun to rise or the user has begun to sweat, the scent circuitry 214 may lessen the amount of scented material dispensed by each pump to avoid overstimulation of the user's senses. As another example, if the sensor data indicates that the user has begun to move her head toward the scent source (e.g., toward a flower in a virtual environment), the scent circuitry 214 may increase the amount of scented material dispensed by each pump to increase scent intensity.

The scent circuitry 214 (which may also be referred to as an “electronics module”) can process data generated by each of the sensor(s) 212 and optimize the delivery of scented material(s) by the pump(s) 204. The scent circuitry 214 can include a power supply (e.g., a battery or an electrical coupling to the power supply of the HMD), memory, one or more processors, and/or a communication module that allows the scent delivery system 202 to communicate with the binocular HMD 200 and/or another computing device (e.g., a mobile phone, tablet, personal computer, or server). The communication module may allow the scent delivery system 202 to communicate over the Internet, a local area network (LAN), a wide area network (WAN), a point-to-point dial-up connection, a cellular network, etc. Additionally or alternatively, the communication module may communicate via a short-range wireless protocol, such as Wi-Fi, near-field communication (NFC), radio-frequency identification (RFID), or Bluetooth.

Scented material(s) are preferably dispensed by the pump(s) 204 responsive to the content is being presented by the binocular HMD 200. For example, the scent circuitry 214 may specify that a floral scent should be produced when a flower is shown in a cinematic video. As another example, the scent circuitry 214 may specify that a pine scent should be produced when a character in non-visual content (e.g., an audiobook) enters a forest. The scent circuitry 214 (as well as the other components of the scent delivery system 202) may be powered through a wired or wireless medium. For example, in some embodiments the scent circuitry 214 includes an internal power supply (e.g., a rechargeable battery or a single cell (i.e., button cell) battery), while in other embodiments the scent circuitry is electrically coupled to, and powered by, the binocular HMD 200.

Some embodiments of the binocular HMD 200 also include one or more heating elements 218 that are disposed near the reservoir(s) 206, pump(s) 204, and/or fan 208. The heating element(s) 218 can be used to dispense the materials stored in the reservoir(s) 206 at certain temperatures or in certain forms. For example, a heating element could be used to produce steam from water stored in a reservoir to increase the humidity within the internal cavity of the structural frame 210. The heating element(s) 218 could also be used to heat an airflow created by the fan 208. For example, a warm airflow may be generated when a desert environment is shown in a cinematic video.

One or more sensors 216 could also be disposed on the outer surface of the scent delivery system 202 and/or the binocular HMD 200. The sensor(s) 216 may monitor various characteristics of the ambient environment. For example, the sensor(s) 216 may include a camera that captures the user interactions with the ambient environment, a light sensor that monitors ambient illuminance levels, an audio sensor that senses vocal commands, etc. One skilled in the art will recognize that other types of sensors could also be included. The type of sensor(s) 216 could be chosen based on whether the HMD system is adapted for a particular application. For example, HMDs designed for outdoor use may include a temperature sensor that generates temperature data and a wind sensor that generates wind data. The temperature data and/or the wind data may be used to determine whether additional scented material needs to be dispensed to counteract a windy environment.

Unlike traditional scent dispersal systems (e.g., the Smell-O-Vision or the Smellitizer), the technologies introduced here are readily portable and allow scents to be delivered directly to an individual experiencing digital content. Said another way, traditional scent dispersal systems are designed to produce scents in a single physical location, while the scent delivery systems described herein can be used regardless of where the individual decides to experience the digital content. Moreover, because the scent can be delivered to a given individual in a targeted manner, other individuals who are nearby will not able to smell the scent (and are thus undisturbed). Consequently, multiple individuals can experience realistic digital content in close proximity to one another despite experiencing different digital content or having different delivery preferences. For example, some individuals may enable the scent functionality while other individuals may opt not to participate in scented experiences.

As shown in FIG. 2A, in some embodiments the binocular HMD 200 includes one or more lighting elements 220 that are installed near the edges of the optical display surface. The lighting element(s) 220 are generally adjustable, thereby enabling the binocular HMD 200 to establish a controlled lighting environment. By intelligently illuminating the lighting element(s) 220, the binocular HMD 200 may make the user believe as though the optical display surface is larger than it truly is. For example, the lighting element(s) 220 could serve as a bias light or backlight by illuminating the periphery of the user's vision based on the visual content being shown (e.g., using a color that matches or complements the visual content). The lighting element(s) 220 could produce white light and/or colored light. The lighting element(s) 220 could also be used to further increase the realism of visual content presented by the binocular HMD 200 in several different respects. For example, the lighting element(s) 220 could provide additional sensory stimuli (e.g., bright bursts of light in the user's peripheral vision during an action movie or shooter video game).

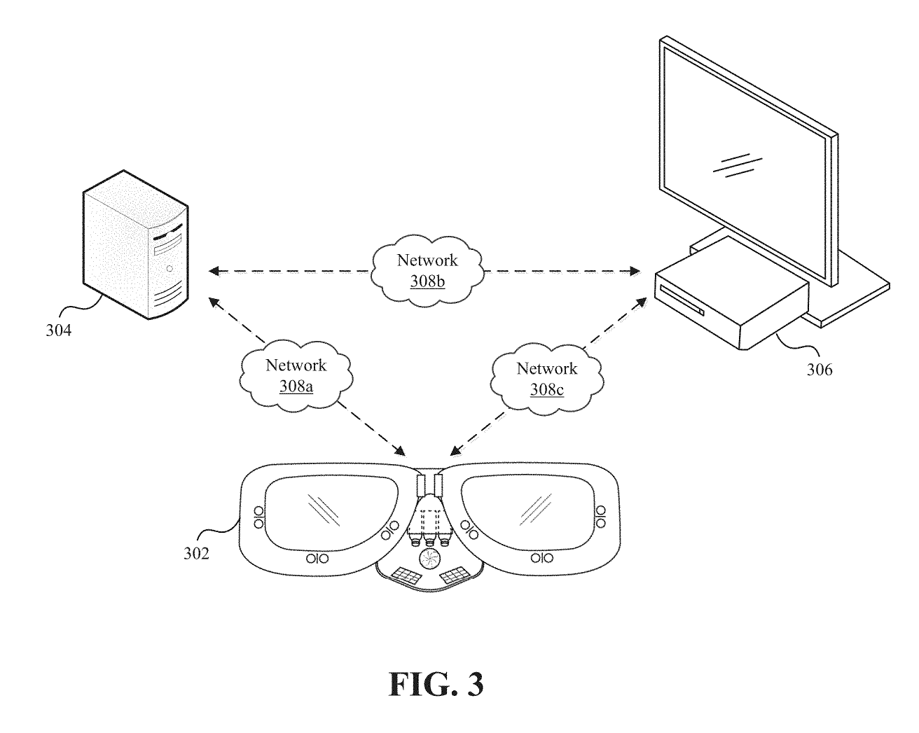

FIG. 3 depicts an example of a network environment that includes an HMD 302 having a scent delivery system, a content server 304, and another computing device 306 (here, a television and game console). The HMD 302, content server 304, and/or computing device 306 can be connected via one or more computer networks 308a-c, which may include the Internet, local area networks (LANs), wide-area networks (WANs), metropolitan area networks (MANs), cellular networks, etc. Additionally or alternatively, the HMD 302, content server 304, and/or computing device 306 may communicate with one another over a short-range communication protocol, such as Bluetooth, near-field communication (NFC), etc. Examples of computing devices 306 include mobile phones, tablet computers, laptop computers, personal computers, network-connected (“smart”) home appliances (e.g., televisions and speakers, such as Amazon Echo® and Google Home®), game consoles (e.g., Sony PlayStation® or Microsoft Xbox® game console), mobile gaming devices (e.g., Sony PSP®), television-connected devices (e.g., Roku®, Apple TV®, Google Chromecast®, Amazon FireStick®, Android TV®, Blu-ray Disc™ players), other network-connected devices having an interface (e.g., fitness trackers, Apple iPod Touch®, Apple Watch®), etc.

Generally, the HMD 302 is responsible for generating and presenting content to a user. While the content is typically in the form of augmented or virtual reality content, other forms of content (e.g., visual content such as cinematic videos and real-time video feeds, or non-visual content such as audiobooks and music) could also be presented to the user. The content server 304 and/or the computing device 306 can be coupled, wired or wirelessly, to the HMD 302. For example, the HMD 302 may be communicatively coupled to the computing device 306 via an wired digital-audio connection (e.g., High-Definition Multimedia Interface (HDMI) or fiber optic), antenna, wireless communication module, etc. In some embodiments the content server 304 and/or the computing device 306 are responsible for delivering content to the HMD 302, while in other embodiments the HMD 302 stores some or all of the content locally (e.g., within a memory).

When the HMD 302 is configured to generate virtual reality content, it may only be communicatively coupled to the content server 304. However, when the HMD 302 is configured to generate augmented reality content, it could be communicatively coupled to the content server 304 and/or the computing device 306. Consequently, the user of the HMD 302 may be able to see augmented reality content when viewing video, listening to audio, or playing video games using the computing device 306. The computing device 306 may also be responsible for presenting content to the user while the HMD 302 simply enhances the realism of the content by generating sensory stimuli (e.g., scents, tastes, and/or visual effects). Thus, the HMD 306 may improve the user experience without presenting content itself.

As noted above, in order to make these experiences more realistic, the HMD 302 (and, more specifically, a scent delivery system) may be configured to produce one or more scents at particular times. For example, the HMD 302 could dispense a floral scent when a digital garden environment is viewable through the HMD 302. As another example, the HMD 302 could dispense a vanilla scent and a cinnamon scent when augmented reality content is viewable through the HMD 302 that augments a cooking show shown on a television in the real world. Further yet, the HMD 302 could dispense a pine scent, a grass scent, or a woody scent when a golf tournament is shown on a television in the real world. In fact, the HMD 302 may switch between these scents as golfers play different holes in different environments. The HMD 302 may produce scents regardless of whether it is responsible for presenting content to the user.

The strength, direction, and frequency of the sensory stimuli produced by the HMD 302 generally correspond to features of the content presented to the user by the HMD 302 and/or the other computing device 306. In some embodiments the instructions for producing the sensory stimuli are produced by a source of the content, while in other embodiments the instructions for producing the sensory stimuli are created automatically (e.g., by the HMD 302, content server 304, or other computing device 306) after analyzing the content to identify elements that are indicative of a sensory stimuli source. The instructions may be embedded within the content (e.g., encoded in a video track, audio track, and/or metadata) or included as a distinct instruction set that accompanies the content and is separately executable by the HMD 302.

In some embodiments, the scent delivery system is housed within a network-connected mask (or some other form of headwear) that allows the individual to view visual content presented by another network-connected computing device, such as a television. The network-connected mask and the network-connected computing device may communicate with one another via a short-range wireless communication technology/protocol, such as Wi-Fi, Bluetooth, NFC, cellular radio, infrared, RFID, etc.

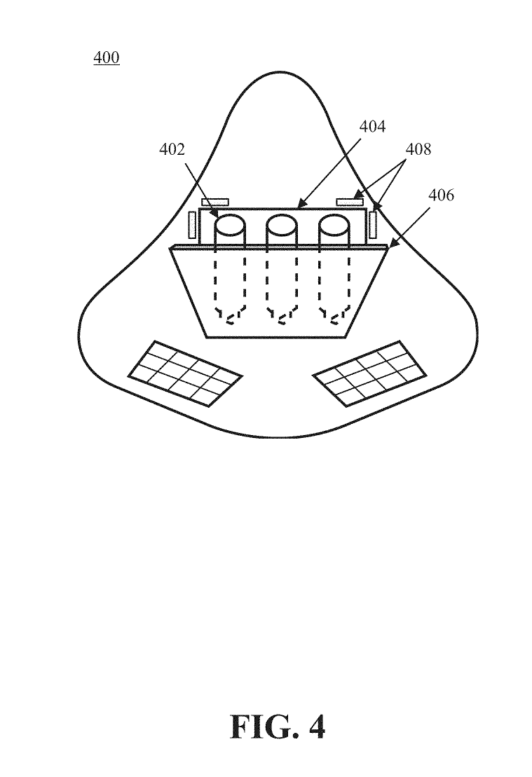

FIG. 4 depicts a scent delivery system 400 that can be detachably connected to an HMD configured to display augmented or virtual reality content. In some embodiments, the scent delivery system 400 includes a compartment 404 that is able to hold one or more reservoirs 402 of scented material (e.g., air or liquid). Each scented material may correspond to a separate “capsule” that is modularly replaceable. Said another way, the capsules can be easily installed within, and removed from, the scent delivery system 400. Each capsules may correspond to a general scent or a specific scent.

As shown in FIG. 4, the reservoirs 402 are often retained within a compartment 404 that is readily accessible to the user while the scent delivery system 400 is attached to an HMD. For example, the scent delivery system 400 may include a door 406 that can be securely closed via fasteners 408, such as magnets, mechanical clips/tracks, an adhesive material/substance, etc. The door 406 enables the user to replace the reservoirs 402 in response to determining new scents are desired or necessary for content that is to be presented by the HMD. For example, the user may wish to replace some or all of the reservoirs 402 prior to watching a cinematic video, playing a video game, or interacting with augmented or virtual reality content. Note, however, that in some embodiments the reservoirs 402 are only accessible when the scent delivery system 400 is separated from the HMD.

The scent delivery system 400 may also dispense scented materials from the reservoirs 402 in order to affect the user's sense of taste. HMDs that show virtual reality content prevent users from observing what they are eating or drinking while viewing the virtual reality content. However, there is a strong physiological connection between different sources of sensory information. For instance, the ability to recognize flavors is often affected by visual information and olfactory information. The technology described herein can make use of these physiological connections to trick the user's senses.

For example, a user could be tricked into believing that water is a sweeter drink by having the HMD present a digital environment that includes a sweet drink and/or by having the scent delivery system 400 produce a sweet scent. Similarly, the user could be tricked into believing a wine is much stronger or better than it actually is by presenting a picturesque vineyard and/or dispensing an appropriate smell. Scent delivery systems can be used to make bland foods seem more salty, spicy, bitter, sweet, or sour by modifying the content presented to the user and/or the scent(s) produced by the scent delivery system 400.

As noted above, the scent delivery system 400 could also store materials that include a flavor compound having a particular taste. Consequently, visual, olfactory, and/or gustatory experiences can be selectively modified in order to improve the user experience. For example, good flavors may be enhanced, while bad flavors may be overwhelmed or replaced entirely. In some embodiments the scent delivery system 400 includes separate reservoirs for storing materials having a scent-enabling compound and materials having a taste-enabling compound, while in other embodiments the scent-enabling compound and the taste-enabling compound are included in the same material that is stored in a single reservoir.

### Implementation Overview

Scented paper products (e.g., fragrance strips) have long been used to increase consumer interest in products such as cosmetics, perfumes, colognes, soaps, shampoos, personal hygiene items, etc. Scent delivery systems are introduced here that are able to bring scented advertisements into other forms of content, including visual content and non-visual content. Such technology can be used to address a fundamental challenge that is inherent in digital transactions (i.e., an inability to accurately gauge different characteristics of a product) by engaging a consumer's senses. More specifically, scent delivery systems can be used to increase the effectiveness of marketing, advertising, and branding campaigns by controllably producing certain scent(s).

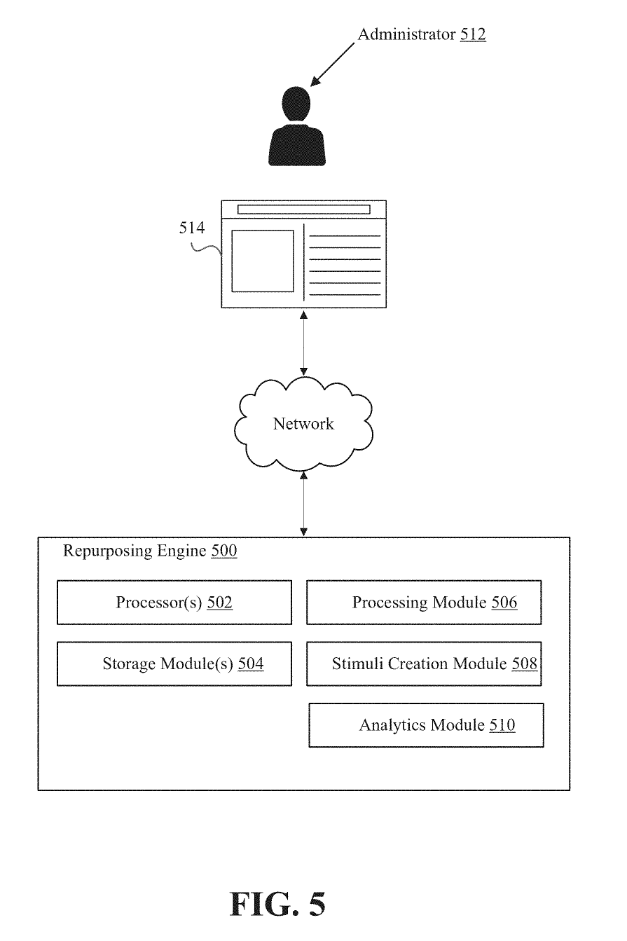

FIG. 5 depicts a development platform 500 that allows content (e.g., visual content and non-visual content) to be specially designed for presentation by a computing device that is connected to a scent delivery system. In such embodiments, a processor can determine whether to dispense a scented material based on instructions created by an administrator 512. The administrator 512 may be associated with a merchant (i.e., manufacturers/sellers of goods/services), a fragrance company (i.e., manufacturers of scented materials), or a development company (e.g., manufacturers of scent delivery systems and producers of digital content). As noted above, the administrator 512 may generate the instructions by working with one or more of these entities to identify the one or more scented materials needed to accurately reproduce a certain scent. The instructions may also specify a delivery timestamp, an intensity/quantity of each scented material, etc. In some embodiments the instructions are embedded within the digital content (e.g., encoded in a video track, audio track, and/or metadata), while in other embodiments the instructions are included in a distinct instruction set that accompanies the digital content and is separately executable by the processor. Similar techniques could also be used for visual content (e.g., applications, games, and cinematic videos) and non-visual content (e.g., audiobooks, concerts, and radio programs).

Older, unscented digital content could also be repurposed for use with a computing device that is connected to a scent delivery system. An administrator 512 may be able to tag unscented digital content to create keyframes that cause the scent delivery system to produce scent(s). For example, the administrator 512 may identify keyframes in a direct advertisement that will benefit from the production of a scent. As another example, the administrator 512 may identify keyframes in existing digital content (e.g., a cinematic film) in order to create an indirect advertisement.

Content modifications may be completed using a graphical user interface (GUI) 514 that is supported by a repurposing engine 500. More specifically, the administrator 512 could upload unscented digital content to the GUI 514, which prompts the repurposing engine 500 to analyze the unscented digital content to determine whether scent(s) should be produced and, if so, which scent(s) should be produced. In some embodiments, the repurposing engine 500 automatically identifies candidate instances for dispensing scented material(s). The candidate instances typically include one or more features (e.g., a character or a product, such as a perfume bottle or a piece of fruit) that are readily recognizable upon performing content analysis. Content analysis may require the repurposing engine 500 execute image processing algorithms, audio processing algorithms, etc. For example, the repurposing engine 500 may perform image segmentation (e.g., thresholding methods such as Otsu's method, or color-based segmentation such as K-means clustering) on individual frames of the digital content to isolate regions and objects of interest. As another example, the repurposing engine 500 may perform audio segmentation on an audio track of the digital content to identify segments that likely correspond with certain events. For instance, significant variations in tempo, pitch, or intensity may be indicative of an event that would benefit from additional sensory stimuli.

In some embodiments, the repurposing engine 500 automatically determines characteristics regarding the production of each scent. The characteristics could include how much scented material should be dispensed (e.g., whether the aroma should be faint, medium, or strong), when the scent material should be dispensed, whether a combination of multiple scented materials is necessary to produce a certain scent, etc. The administrator 512 may be able to modify the instances and/or characteristics proposed by the repurposing engine 500. The administrator 512 may also be able to specify additional instances and/or characteristics using the GUI 514.

Thus, unscented digital content could be manually repurposed into scented digital content by an administrator 512 and/or automatically repurposed into scented digital content by the repurposing engine 500. The unscented digital content could be non-visual content or visual content, such as two-dimensional (2D) content or three-dimensional (3D) content. The repurposing engine 500 may also engage in machine learning to identify candidate instances and keyframes, identify the appropriate scent(s) to be produced at the keyframes, etc. For example, the repurposing engine 500 may monitor the overall effectiveness of a scent marketing campaign by analyzing different quantitative metrics, such as the merchant's retail traffic, click-through rates, conversion rates, etc. Such techniques allow the repurposing engine 500 to identify the characteristics (e.g., scent type, scene type, and content type) that correspond with effective scent marketing campaigns, and then use this information to more intelligently produce scents that align with content. For example, the repurposing engine 500 may discover that scented advertisements for a perfume product are much more effective when shown after romance films rather than action films or comedy films.

The repurposing engine 500 includes one or more processors 502 for executing these tasks and one or more storage modules 504 for storing the relevant data (e.g., digital content, ground truth feature sets for identifying candidate instances, known combinations of scented materials). The tasks performed by the repurposing engine 500 could also be performed by a series of dedicated modules. For example, a processing module 506 may be responsible for identifying candidate instances, a stimuli creation module 508 may be responsible for identifying the scented material(s) needed to produce a certain scent, and an analytics module 510 may be responsible for monitoring the effectiveness of a scent marketing campaign, noting user reactions to different scents, etc.

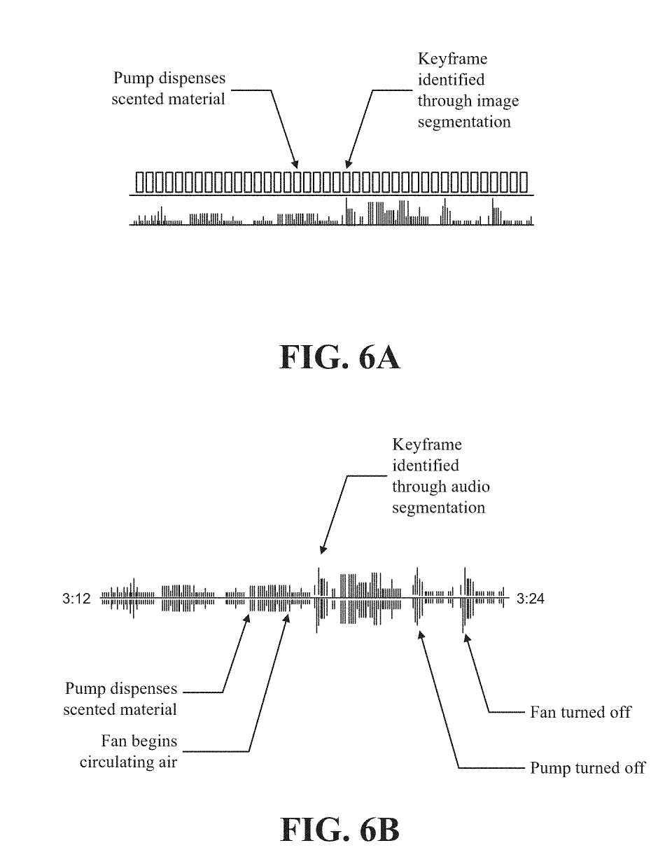

Disjointed experiences will often occur if scented are produced too early or too late. Accordingly, the repurposing engine 500 may employ predictive analytics to determine when, exactly, a scent should be produced for a given keyframe. FIG. 6A depicts an example of a video track that includes a keyframe identified through image segmentation. FIG. 6B, meanwhile, depicts an example of an audio track that includes a keyframe identified through audio segmentation. As shown here, predictive analytics models may prompt a scent delivery system to begin producing a scent before the keyframe is shown or heard. For example, the lead time could be measured by duration (e.g., 50 milliseconds) or frame count (e.g., 5 frames). In some embodiments, the lead time is based on a user characteristic known to affect sensory sensitivity, such as age, gender, relevant ailments (e.g., infections and injury), smoking status, etc.

However, the lead time could also vary based on other factors, such as the type of digital content, the duration of usage of the scent delivery system, etc. For example, the lead time may increase proportional to the duration of usage. Long usage durations generally correspond to decreases in sensory sensitive and increases in reaction time (i.e., the elapsed time between the presentation of a sensory stimulus and the subsequent behavioral response). As another example, the lead time may increase for some types of digital content (e.g., visual content where multiple senses are engaged) and decrease for other types of digital content (e.g., non-visual content where only the sense of hearing is engaged). Predictive analytics models may also be used to determine when the scent delivery system should cease production of scent, airflow, heat, etc.

Keyframes may be used to indicate when various events should occur. FIG. 6A, for example, illustrates how a keyframe may prompt a scent delivery system to perform a single action (e.g., eject a scented material). However, a keyframe could also induce multiple actions. FIG. 6B, for example, illustrates how a keyframe may prompt a scent delivery system to begin ejecting a scented material, begin circulating air, cease ejecting the scented material and cease circulating air. An individual keyframe could also be used to induce each event such that multiple actions correspond with multiple keyframes, though keyframes could be shared.

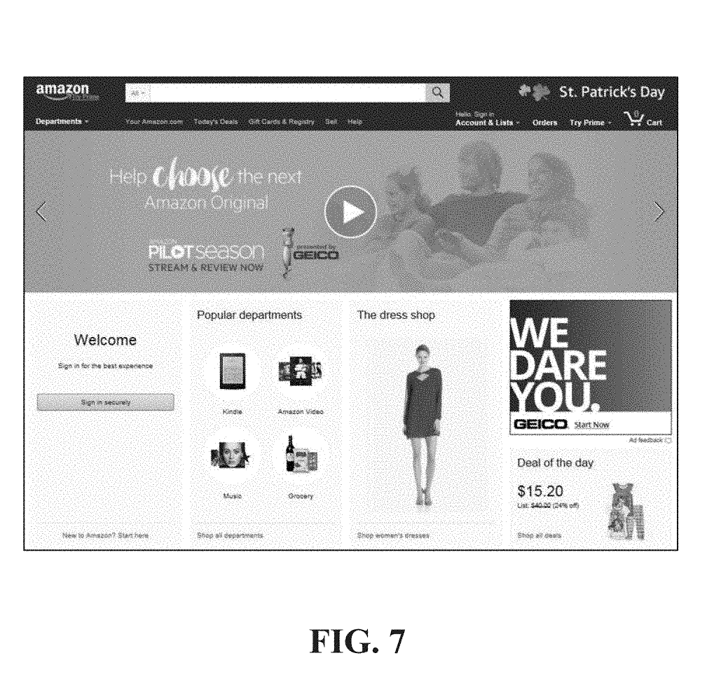

FIG. 7 depicts an interface through which a user can browse items offered for sale by an electronic commerce platform, which could range in size from large retailers (e.g., Amazon.com®) that sell a wide assortment of products to small retailers that sell a single product. The interface may be viewed by the user on an HMD or some other computing device (e.g., computing device 306 of FIG. 3). A scent delivery system that is communicatively coupled to the HMD or the other computing device may enable scent-based browsing of the items offered for sale.

For example, a user may execute a search for perfumes or colognes through the interface, and then browse different items returned in the search result. Similarly, the user might visit a virtual perfume shop or virtual cologne shop where the user is able to view and smell different items offered for sale. As another example, the user could browse scents and purchase a scent (e.g., as part of a gift that is paired with a cinematic movie, digital card, etc.) that will be experienced by the user or another user.

A scent delivery system may permit the user to test the scents of different physical items (e.g., perfumes or colognes) before placing an online order for a physical item. Said another way, a scent delivery system may enable scent-based browsing of products that are available for purchase by the user. Although the scent of a perfume or cologne may not be replicated exactly, the scent(s) produced by a scent delivery system enable the user to have a better understanding of what she is buying (e.g., whether a perfume or cologne is intended to have a sweet fragrance, a floral fragrance, a woody fragrance, etc.).

As noted above, scent delivery systems produces scent(s) upon executing instructions that correspond to the content being experienced by the user. The instructions could be initially created in several different ways. For example, a manufacturer (e.g., a perfume manufacturer) may select one or more scents from a list of scents that are commonly produced by scent delivery systems. As another example, instructions could be automatically created by parsing a product listing to identify tag words that are used to describe the item. For example, a repurposing engine (e.g., repurposing engine 500 of FIG. 5) may execute speech recognition techniques, such as keyword spotting, to detect tag word(s) that may be used to identify the scent(s) that are most appropriate. One skilled in the art will recognize that these different techniques for creating instructions enable different levels of control in producing scents and accuracy in replicating aromas. For instance, instructions provided by the manufacturer of the item are generally more accurate than instructions that are automatically created based on available information about the item.

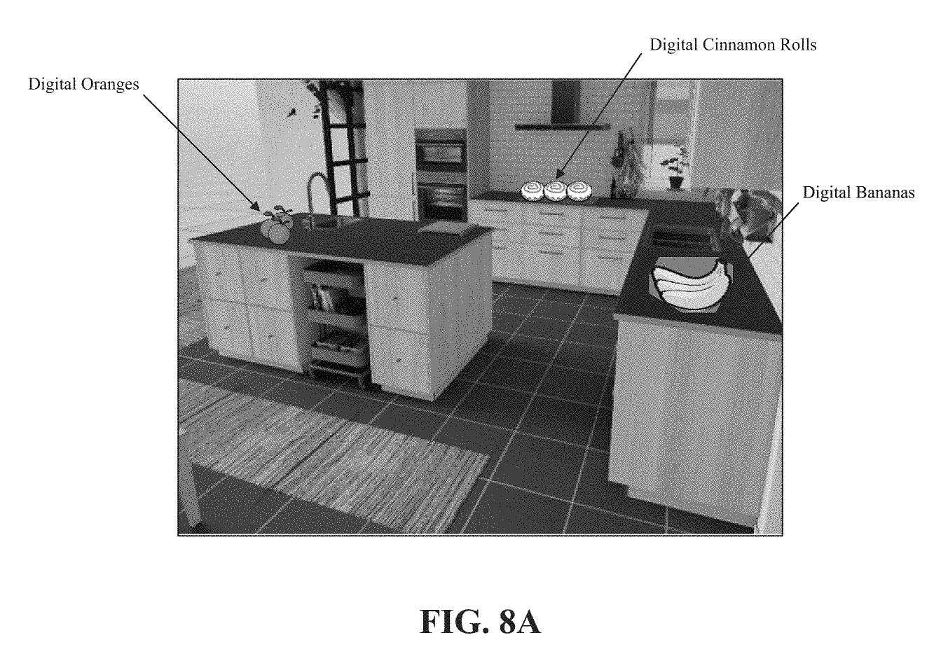

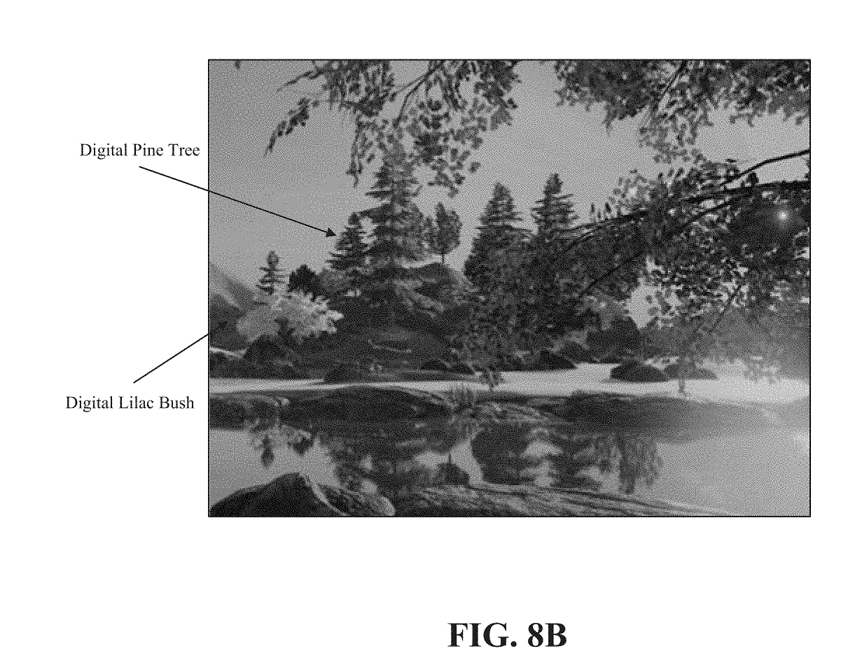

FIG. 8A depicts a digital kitchen environment that is viewable through an HMD. FIG. 8B, meanwhile, depicts a digital garden environment that is viewable through an HMD. One skilled in the art will recognize that these digital environments have been selected for the purpose of illustration only.

A scent delivery system that is connected to the HMD can be configured to dispense certain scented material(s) as the user of the HMD traverses the digital environments. These digital environments may be traversed over the course of a cinematic film, video game, etc. For example, as the user navigates the digital kitchen shown in FIG. 8A and approaches the digital oranges, the scent delivery system may produce a citrus scent. As another example, as the user navigates the digital garden shown in FIG. 8B and approaches the digital lilac bush, the scent delivery system may produce a lilac scent or a floral scent. These scents may be produced before the user is able to visualize the corresponding digital features.

Much like fragrance strips that are placed inside magazines, these scent(s) can be used as part of a scent-based advertising campaign. Several different use cases illustrate the benefits of enhancing the realism of digital content (e.g., visual content and non-visual content) by replacing unrelated real-world stimuli with controlled stimuli.
Direct Advertising: A scent delivery system can produce an appropriate scent while an individual watches an advertisement for an item (e.g., a perfume or a deodorant). The individual may also be permitted to buy the item in real time during the advertisement. For example, if the individual exhibits an interest in the scent (e.g., by moving her head closer to the digital source of the scent), the individual may be prompted to decide whether she would like to order the item directly through the advertisement. That is, the individual may be able to complete a digital transaction without disrupting the current experience by navigating to a separate channel.
Indirect Advertising: Scent(s) related to digital content experienced by an individual can be intelligently replicated by a scent delivery system, which allows products already present in the digital content to be indirectly advertised to consumers. For example, the scent delivery system may produce a perfume scent when a female character in a cinematic film approaches the camera. The individual may be able to order the item in real time (i.e., as the digital content continues to run) and/or save a product summary for subsequent ordering. Moreover, a list of product(s) indirectly advertised throughout the runtime of the digital content could be shown to the individual following the conclusion of the digital content. In such instances, the individual may prompt the scent delivery system to reproduce scent(s) that were previously produced during the runtime of the digital content. Indirect advertising techniques are particularly useful for those forms of content where direct advertising is undesirable (e.g., due to content flow disruptions) or difficult to carry out (e.g., due to the presence of ad blockers).
Business Model Improvements: Merchants can work with fragrance companies and development companies to re-create scents that will improve the effectiveness of advertising campaigns, the desirability of products, etc. For example, some or all of these entities may work together to develop a scented advertisement and generate instructions for producing the appropriate scent(s) at the appropriate time(s).
Promotional Cartridges: Merchants could send promotional capsules that include scented materials out on a periodic (e.g., weekly or monthly) basis. For example, a perfume manufacturer may distribute promotional capsules each time a new perfume is developed. As another example, the perfume manufacturer may distribute promotional capsules as part of the concept testing process that typically occurs prior to production (e.g., multiple capsules could be delivered to potential consumers who are asked to identify their favorite scent or rate the various scents).

HMDs often employ an optical positional tracking technique (e.g., inside-out tracking and outside-in tracking) to determine the position of certain objects, such as a user's hands and feet. Accordingly, certain scent(s) may be triggered by the user as she navigates the digital environment. For example, a scent delivery system may produce a citrus scent responsive to a determination that the user has reached out her hand and grasped at the digital oranges. As another example, a scent delivery system may produce a strawberry scent responsive to a determination that the user has stepped on a digital strawberry.

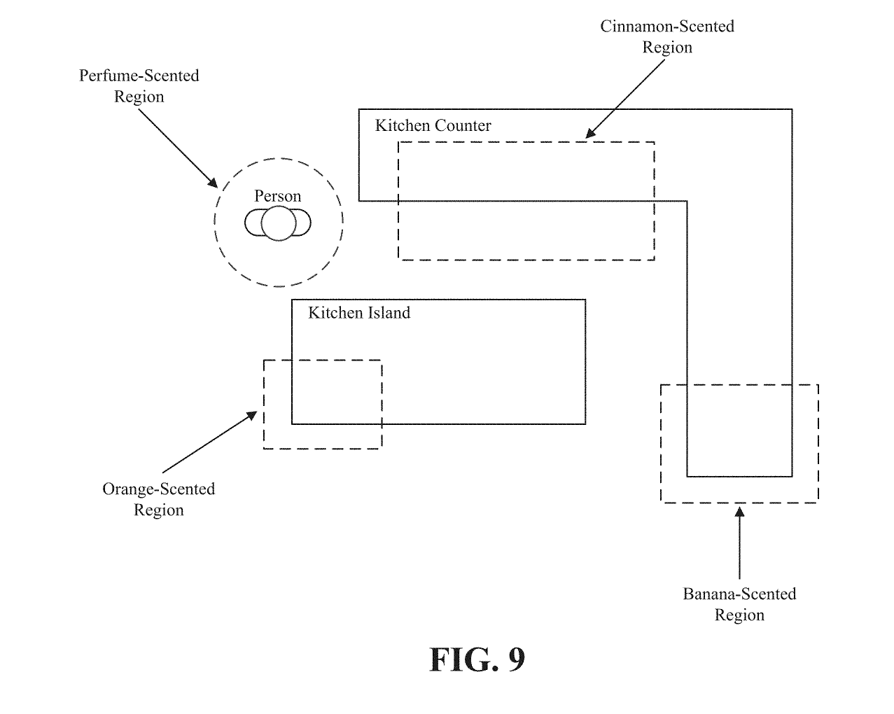

FIG. 9 includes an overhead map illustrating how a digital environment may include one or more scented regions. The overhead map shown here largely corresponds to the digital kitchen shown in FIG. 8A. As shown here, the scented region(s) will often vary in size and/or shape. In some embodiments these parameters are automatically determined based on the scent type or certain user characteristics (e.g., known sensitivities to certain scent types), while in other embodiments these parameters are manually specified by an administrator who is responsible for identifying the scented features. Thus, users may be able to smell items before those items are shown or described (which can be used as another form of indirect advertising).

Some of the scented region(s) may be locked in a static (i.e., fixed) location, while other scented region(s) may move throughout the digital environment. For example, the perfume-scented region may move as the digital person traverses the digital kitchen environment. Consequently, scented regions may occasionally overlap. A scent delivery system will typically produce both scents in such instances, although the scent delivery system may also produce a single scent in some instances (e.g., where one scent is likely to overpower another).

Scented region(s) may also be accessible for a specified duration of time. For example, the orange-scented region may disappear if the digital oranges are eaten or thrown in the trash. As another example, the cinnamon-scented region may disappear upon determining the digital cinnamon rolls have been shown for a certain period of time. Therefore, an individual may smell different scents while traversing a digital environment. The individual may even begin to smell items before those items are viewable within the digital environment. Consequently, different scents may be used to guide the individual through the digital environment. For example, a favorable scent may attract to the individual to a certain room within the digital environment, and the intensity of the favorable scent may increase as the individual moves closer to the certain room.

Although the overhead map of FIG. 9 represents a digital kitchen environment, the same techniques are equally applicable to other digital environments. For example, the technology introduced here may allow an individual to traverse a digital recreation of a merchant's physical store and smell different products that are offered for sale by the merchant. Smell has been shown to be particularly effective in marketing products such as cosmetics, perfumes, colognes, soaps, shampoos, personal hygiene items, etc.

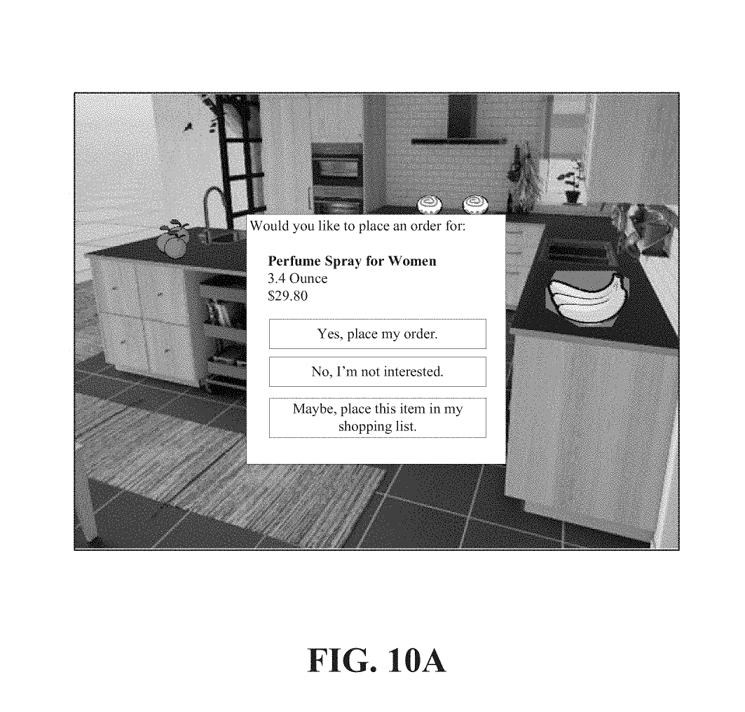

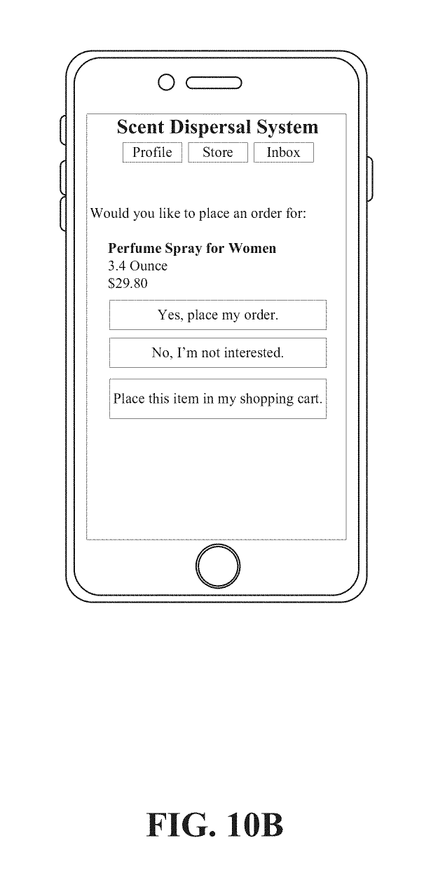

If an individual indicates an interest in a product (e.g., by moving her head closer to the digital source of the scent), the individual may be asked whether she wishes to purchase the product. This can occur in several different ways. For example, as shown in FIG. 10A, an order interface may be presented by the computing device (e.g., an HMD or a network-connected television) that is used to view the visual content. The order interface may prompt the user to approve a purchase of the product, input financial information (e.g., name, address, and payment card details), decline a purchase of the product, or add the product to a queue (often referred to as a “shopping cart”).

Additionally or alternatively, as shown in FIG. 10B, the order interface may be presented by another computing device that is communicatively coupled to the computing device used to view the visual content. For example, the individual may receive notifications on a mobile phone while viewing the visual content on an HMD or a network-connected television. The order interface may be delivered through any suitable communication channel (e.g., as a user interface pop-up, push notification, text message, or email message).

One skilled in the art will recognize that an individual may indicate an interest in a product in many different ways. For example, the individual may move her head toward the digital source of the scent, traverse the same location within a digital environment multiple times in order to re-smell the scent, or perform a certain gesture (e.g., a grabbing motion) or action (e.g., pressing a mechanical button on a control device) that is indicative of an interest in the product.

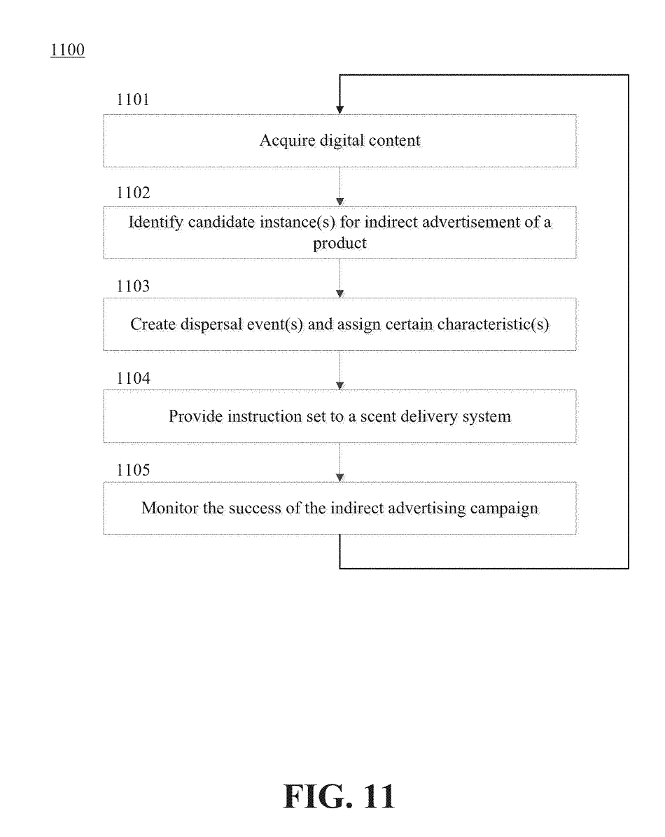

FIG. 11 depicts a process 1100 for generating an instruction set for a scent delivery system. Digital content is initially received by a repurposing engine (step 1101). For example, the digital content may be manually uploaded by an administrator through a GUI (e.g., GUI 514 of FIG. 5). As another example, the digital content may be automatically retrieved by the repurposing engine from a content database that is hosted on a local computing device (i.e., on the same computing device as the repurposing engine) or a remote computing device that is accessible over a network. The administrator may be associated with a merchant, a fragrance company, or a development company.

The repurposing engine can then identify one or more candidate instances for an advertisement of a product (step 1102). In some embodiments, the repurposing engine parses the digital content to automatically identify the candidate instance(s) on behalf of the administrator. For example, the repurposing engine may apply image processing algorithms to identify characters, items, etc., that are embedded within the digital content. More specifically, the repurposing engine may perform image segmentation (e.g., thresholding methods such as Otsu's method, or color-based segmentation such as K-means clustering) on individual frames of visual content to isolate regions and objects of interest. In other embodiments, the repurposing engine identifies the candidate instance(s) upon receiving user input from the administrator. For example, the administrator may manually specify that a particular character or item is shown in a given frame of the digital content.

A dispersal event can then be created by the repurposing engine based on characteristic(s) that are automatically specified by the repurposing engine or manually specified the administrator (step 1103). The characteristic(s) can include, for example, which scented material(s) should be dispensed, scent duration, scent intensity, etc. Such characteristic(s) are typically specified by the administrator, who may work with a merchant, a fragrance company, or development company to identify which scented material(s) are required to increase the desirability of the product. Dispersal events are often offset from the corresponding candidate instances in order to avoid the creation of disjointed experiences where scent production does not align with audio or video. The lead time could be measured by duration (e.g., 50 milliseconds) or frame count (e.g., 5 frames). In some embodiments the lead time is fixed, while in other embodiments the lead time is variable. For example, lead times may be increased if a series of dispersal events (and thus a series of scents) are to be experienced quickly in succession.

An instruction set that includes the dispersal events can be generated for the digital content and provided to a scent delivery system (step 1104). In some embodiments the instructions are embedded within the digital content (e.g., encoded in a video track, audio track, and/or metadata), while in other embodiments the instructions are included in a distinct instruction set that accompanies the digital content and is separately executable by the scent delivery system. Although the process 1100 is described using visual content as an example, a similar technique could be used to create dispersal events for non-visual content (e.g., audiobooks, concerts, and radio programs).

In some embodiments, an analytics module monitors the success of an advertising campaign by observing user reactions to the scented advertisement (step 1105). The analytics module may be executed by the repurposing engine or a separate engine (in which case the repurposing engine and the analytics module may be hosted on different computing devices). The analytics module may monitor the overall effectiveness of a scent marketing campaign by analyzing different quantitative metrics, such as traffic statistics for the merchant's website, the merchant's retail traffic, click-through rates, conversion rates, etc. Such techniques allow the analytics module to identify the features that correspond with effective scent marketing campaigns, such as the dispersal timing, scent duration, scent intensity, scent type, scene type, and content type. The repurposing engine can then use this information to more intelligently produce scent(s) that align with content and lead to improvements in user engagement. For example, the analytics module may discover that scented advertisements for a perfume product are much more effective when shown before, during, or after romance films rather than action films or comedy films.

Various machine learning algorithms and techniques could be employed by the advertising engine, including Naïve Bayes Classifier algorithms, K Means Clustering algorithms, Support Vector Machine algorithms, linear regression, logic regression, artificial neural networks, etc. These machine learning algorithms/techniques may be chosen based on application (e.g., supervised or unsupervised learning) and optimized based on whether an administrator confirmed/denied candidate instances that were proposed by the repurposing engine, indicated that a certain scent created a more realistic or desirable experience, indicated that a certain scent led to more positive advertising outcomes (e.g., increased sales or traffic), etc.

The process 1100 can be used to create direct advertisements or indirect advertisements. However, because direct advertisements need not be subtly embedded within the digital content, it is typically much easier to identify when a direct advertisement has begun and when an individual should be prompted to purchase the product (e.g., at the conclusion of the direct advertisement).

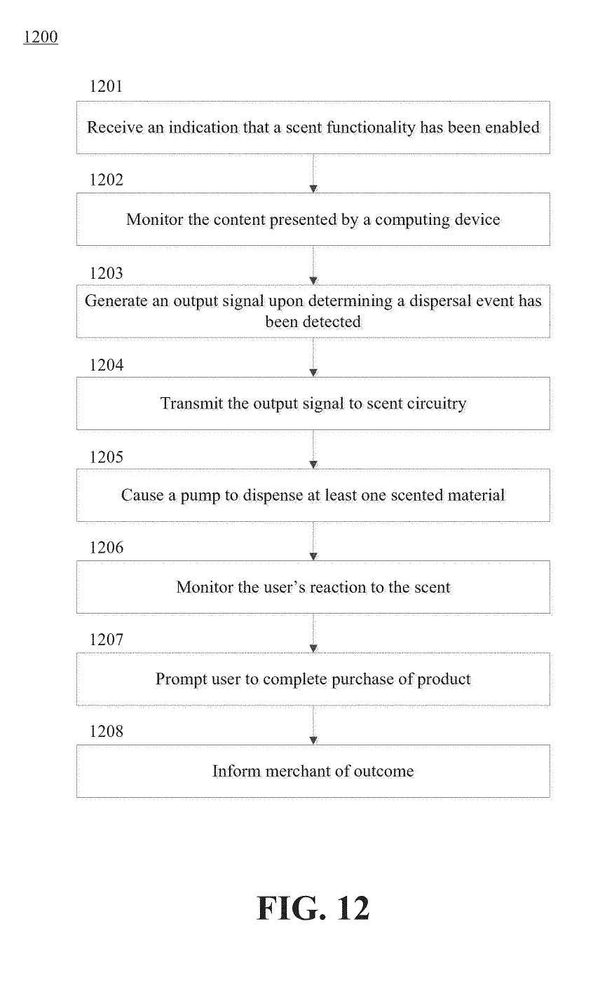

FIG. 12 depicts a process 1200 for controllably producing scents that correspond with digital content experienced by a user. More specifically, a scent delivery system can dispense one or more scented materials while the user experiences the digital content (e.g., visual content or non-visual content). The scent delivery system may be connected to a computing device that is responsible for presenting the digital content. Initially, scent circuitry of the scent delivery system receives an indication that a scent functionality has been enabled (step 1201). The scent functionality could be enabled using a mechanical switch on the scent delivery system or an electronic interface presented by the computing device. In some embodiments, the scent functionality is automatically enabled upon determining the scent delivery system is connected to the computing device unless the user specifies otherwise. The scent circuitry can include a power supply, memory, processor(s), and/or communication module(s) that enable the scent delivery system to communicate with the computing device across a wired or wireless communication channel.

The scent delivery system monitors the digital content presented by the computing device (step 1202). The digital content could be visual content (e.g., cinematic videos, applications, or video games) or non-visual content (e.g., audiobooks, concerts, and radio programs). The scent delivery system can then generate an output signal in response to determining a dispersal event has been detected (step 1203). The dispersal event corresponds to a keyframe (e.g., a video frame or an audio frame) that includes or makes reference to a particular product. Dispersal events cause the scent delivery system to produce scents that are related to the product included in the keyframes. However, dispersal events are often offset from the corresponding keyframes so that the different sensory stimuli (e.g., smell, taste, sight, and/or hearing) are substantially synchronized. The term “keyframe” applies regardless of whether the content is visual or non-visual. For example, both cinematic videos and audiobooks can have keyframe(s) arranged throughout their runtime.

The output signal could also include other metadata about the scent that is to be produced. For example, the output signal could specify which pump(s) should be activated and the amount of scented material that should be dispensed by each pump. Output signals could also be generated that induce activation/deactivation of a fan (e.g., fan 208 of FIG. 2), a heating element (e.g., heating element 218 of FIG. 2), certain sensors (e.g., sensors 212 or sensors 216 of FIG. 2), or certain lighting elements (e.g., lighting elements 220 of FIG. 2). The output signals (and any corresponding metadata) could be modified based on criteria such as age, gender, lifestyle, usage statistics, etc. For example, scent intensity may be increased proportional to the age of the user or decreased proportional to the current duration of usage.

Typically, the output signal is transmitted to the scent circuitry of the scent delivery system (step 1204). The scent circuitry processes the output signal in order to determine which scented material(s) should be dispensed, how much of each scented material should be dispensed, etc. The scent circuitry can then cause pump(s) to controllably dispense the scented material(s) toward the user's nose (step 1205). The scented material(s) are typically intended to produce a scent that mimics the actual scent of digital content that is included in the keyframe. However, in some instances the scent simply increases the realism or desirability of the digital content itself. For example, an advertisement for an athletic event or a movie may be accompanied by a popcorn scent or a caramelized sugar scent.

The amount of scented material that is released by each pump may correspond to the digital content being presented by the computing device. For example, additional scented material may be dispensed as the user approaches the digital source of a scent. Thus, the scent intensity may increase as the user approaches the digital source. As another example, a pump may dispense less scented material if the user is concurrently experiencing visual stimulation that is related to the scent. Said another way, smaller amounts of scented material may be required when the user is also expected to experience significant visual stimulation and/or audible stimulation. For instance, a faint lemon aroma may be sufficient if a digital bowl of lemons is readily viewable, while a strong lemon aroma may be necessary if the user is walking through a digital citrus grove whose fruit is not easily viewable. Realistic simulation of visual content requires that the strength, direction, frequency, and other features of any sensory stimuli must generally correspond to what is being presented by the computing device. Accordingly, the scent circuitry may consider what each of the user's other senses are expected to experience at a given point in time.

The scent delivery system and/or the computing device can also be configured to monitor the user's reaction to the scent (step 1206). In some embodiments, the computing device and/or the scent delivery system include temperature sensor(s) that monitor whether the user's temperature has begun to rise and/or humidity sensor(s) that monitor whether the user has begun to sweat. Both of these outcomes may be indicative of overstimulation of the user's senses. Similarly, the computing device and/or the scent delivery system may include motion sensor(s) that are able to determine whether the user has begun to move her head away from the digital source of the scent or has begun to shake her head. These actions may indicate that the scent produced by the scent delivery system is too weak or strong. A fan may circulate the air around the user's nose or a pump may dispense a neutralizing scent to eliminate or dilute strong scents.

If the user's reaction is indicative of an interest in the product shown or referenced in the keyframe, the user may be asked whether she wishes to purchase the product (step 1107). This can occur in several different ways. In some embodiments, a notification may be presented by the computing device used to view the visual content. As shown in FIGS. 10A-B, the notification may prompt the user to approve a purchase of the product, input financial information (e.g., name, address, and payment card details), decline a purchase of the product, or add the product to a queue (often referred to as a “shopping cart”) for further review. In other embodiments, a notification may be presented by another computing device that is communicatively coupled to the computing device used to view the visual content. For example, the individual may receive notifications on a mobile phone while viewing visual content on an HMD or a network-connected television.

Responsive to a determination that the user has asked to place an order for the product, the scent delivery system can inform a merchant who facilitates completion of the transaction (step 1108). In some instances, the financial information needed to complete the transaction may be readily available to the merchant. For example, the merchant may already have such information on file from previous transactions with the user. As another example, the scent delivery system (or another computing device operated by the user) may be able to access such information and provide it to the merchant.

Note, however, that scents need not always provoke order interfaces, even if those scents are intended to serve as scented advertisements. Such scenarios are another form of indirect advertising. For example, scents produced during the runtime of the digital content may correspond to a list of product(s) that are available for purchase. But the list of product(s) may only be presented following to conclusion of the digital content or upon request from the user.

Unless contrary to physical possibility, it is envisioned that the steps described above may be performed in various sequences and combinations. For example, in some embodiments the process for identifying candidate instance(s) and/or specifying the characteristic(s) of each dispersal event involves significant administrator input. In fact, targeted advertising campaigns may require that developers work closely with merchants, fragrance companies, and/or development companies to identify the most appropriate scented materials, dispersal times, scent intensities, etc. Consequently, specific sets of user interfaces may be used to generate the instruction sets that are executed by the scent delivery systems to produce scents.

Additional steps could also be included in some embodiments. For example, the scent delivery system may require that the user opt in or opt out of scent-based advertising campaigns. Moreover, other stimuli (e.g., gustatory or tactile, such as simulated wind) could also be used as a component of a direct advertising campaign or an indirect advertising campaign. For example, a direct advertising campaign for a coastal vacation resort may prompt the scent delivery system to produce a salty scent and create a varied air flow that is intended to simulate gusts of wind.

### Emotion Manipulation

Conventional forms of digital content often use sound to manipulate an individual's emotions. Examples of panic noises can be found in thriller films and horror films, which often employ dissonant, screeching sounds that viewers unconsciously associate with animals in distress. In fact, a study by the University of California found that human sensitivity to non-linear alarm sounds, such as those made by groundhogs to warn about predators, is employed by film composers to unsettle and unnerve. Music is often employed with the intention that is accomplish many of the same goals. For example, Psycho by Alfred Hitchcock includes straining strings and overblowing brass that mimic a panicky noise.

The relationship between sound and mood or emotional context is often used to guide individuals through digital content. For example, film scores often “build up” as the intensity of a situation rises, while the musical climaxes of film scores often coincide with crucial plots events, both good and bad.

Scents have been shown to affect mood, work performance, and behavior in a similar manner. However, in order for a scent to elicit a response, an individual will typically have to first learn to associate the scent with some type of event. The process by which one event or item comes to be linked with another because of a past experience is referred to as “associative learning.” The linked event is then able to elicit a conditioned response for the original situation. For example, a novel scent may be experienced in the context of an unconditioned stimulus (e.g., procedure in a dental clinic or hospital) that elicits an unconditioned emotional response, such as anxiety. The scent then becomes a conditioned stimulus for that experience and acquires the ability to elicit the conditioned emotional response when encountered in the future.

Scent delivery systems can make use of these mechanisms to manipulate mood and emotion. More specifically, scent delivery systems can produce scents (or other sensory stimuli) that elicit certain emotions from individuals experiencing digital content.

Studies have shown that prosocial behaviors and outlook/attitude are often enhanced in the presence of pleasant scents. Accordingly, a scent delivery system may produce a pleasant scent as the digital content approaches an event having a positive outcome. The pleasant scent is intended to elicit a conditioned emotional response (e.g., happiness) by an individual, who may have smelled a prior production of the same scent or a similar scent. For example, the same pleasant scent could be used throughout the digital content. As another example, the pleasant scent may mimic a scent (e.g., cotton candy or caramelized sugar) that is often associated with positive experiences.

Improvements in mood have been linked to increases in productivity, performance, and a tendency to help others. Thus, scent production may be particularly useful for scent delivery systems that can be used while users interact with the real world, such as those connected to HMDs configured to present augmented reality content. For example, a scent delivery system may produce a pleasant scent (e.g., coffee or caramelized sugar) that increases the user's real-world productivity and performance.

Studies have also shown that unfavorable scents can lead to a negative mood that reduces prosocial behavior. Accordingly, a scent delivery system may produce an unfavorable scent as the digital content approaches an event having a negative outcome in order to build suspense, anxiety, fear, sadness, joy, etc. Examples of unfavorable scents include smoke, rotting flesh, and ammonia.

For example, a scent delivery system may produce a mud scent during an advertisement for a security system. The mud scent may be accompanied by an air flow intended to simulate wind and water droplets intended to simulate rain. The air flow may be created by the fan, while the water droplets may be controllably dispensed by a pump. The strength, direction, frequency, and temperature of the air flow or water droplets may correspond to features of the digital content presented to an individual.

Together, these sensory stimuli may be sufficient to increase the intensity of certain emotional responses. Note, however, that scents can be used to evoke other feelings as well. For example, food-based scents may be used to heighten a sensation of hunger. In some embodiments, emotions are further influenced by the dispersal of certain pheromone(s) intended to trigger specific response(s), such as alarm pheromones, signal pheromones, sex pheromones, etc.

### Processing System

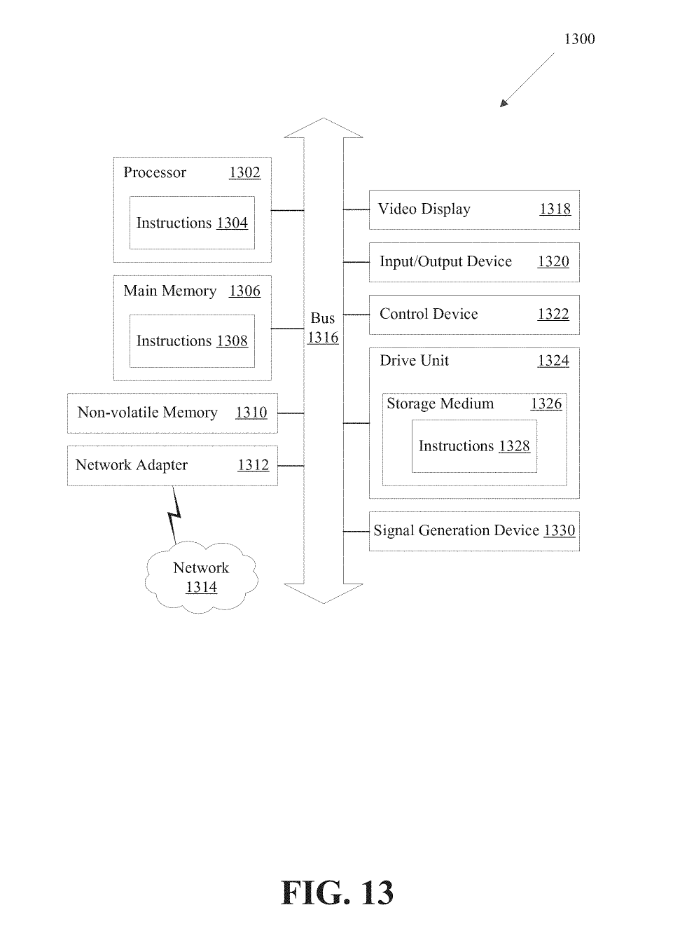

FIG. 13 is a block diagram illustrating an example of a processing system 1300 in which at least some operations described herein can be implemented. The processing system may include one or more central processing units (“processors”) 1302, main memory 1306, non-volatile memory 1310, network adapter 1312 (e.g., network interfaces), video display 1318, input/output devices 1320, control device 1322 (e.g., keyboard and pointing devices), drive unit 1324 including a storage medium 1326, and signal generation device 1330 that are communicatively connected to a bus 1316. The bus 1316 is illustrated as an abstraction that represents one or more physical buses and/or point-to-point connections that are connected by appropriate bridges, adapters, or controllers. The bus 1316, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus (also referred to as “Firewire”).

In some embodiments the processing system 1300 operates as part of an HMD (e.g., HMD 200 of FIG. 2), while in other embodiments the processing system 1300 is connected (wired or wirelessly) to the HMD or some other computing device. In a networked deployment, the processing system 1300 may operate in the capacity of a server or a client machine in a client-server network environment, or as a peer machine in a peer-to-peer network environment.

The processing system 1300 may be a server, a personal computer (PC), a tablet computer, a laptop computer, a personal digital assistant (PDA), a mobile phone, a processor, a telephone, a web appliance, a network router, a switch, a bridge, a console, a gaming device, a music player, or any machine capable of executing a set of instructions (sequential or otherwise) that specify actions to be taken by the processing system 1300.

While the main memory 1306, non-volatile memory 1310, and storage medium 1326 (also called a “machine-readable medium”) are shown to be a single medium, the term “machine-readable medium” and “storage medium” should be taken to include a single medium or multiple media (e.g., a centralized or distributed database, and/or associated caches and servers) that store one or more sets of instructions 1328. The term “machine-readable medium” and “storage medium” shall also be taken to include any medium that is capable of storing, encoding, or carrying a set of instructions for execution by the processing system 1300.

In general, the routines executed to implement the embodiments of the disclosure may be implemented as part of an operating system or a specific application, component, program, object, module or sequence of instructions referred to as “computer programs.” The computer programs typically comprise one or more instructions (e.g., instructions 1304, 1308, 1328) set at various times in various memory and storage devices in a computing device, and that, when read and executed by the one or more processors 1302, cause the processing system 1300 to perform operations to execute elements involving the various aspects of the disclosure.

Moreover, while embodiments have been described in the context of fully functioning computing devices, those skilled in the art will appreciate that the various embodiments are capable of being distributed as a program product in a variety of forms. The disclosure applies regardless of the particular type of machine or computer-readable media used to actually effect the distribution.

Further examples of machine-readable storage media, machine-readable media, or computer-readable media include, but are not limited to, recordable-type media such as volatile and non-volatile memory devices 1310, floppy and other removable disks, hard disk drives, optical disks (e.g., Compact Disk Read-Only Memory (CD ROMS), Digital Versatile Disks (DVDs)), and transmission-type media such as digital and analog communication links.

The network adapter 1312 enables the processing system 1300 to mediate data in a network 1314 with an entity that is external to the processing system 1300 through any communication protocol supported by the processing system 1300 and the external entity. The network adapter 1312 can include one or more of a network adaptor card, a wireless network interface card, a router, an access point, a wireless router, a switch, a multilayer switch, a protocol converter, a gateway, a bridge, bridge router, a hub, a digital media receiver, and/or a repeater.

The network adapter 1312 can include a firewall that governs and/or manages permission to access/proxy data in a computer network, and tracks varying levels of trust between different machines and/or applications. The firewall can be any number of modules having any combination of hardware and/or software components able to enforce a predetermined set of access rights between a particular set of machines and applications, machines and machines, and/or applications and applications (e.g., to regulate the flow of traffic and resource sharing between these entities). The firewall may additionally manage and/or have access to an access control list that details permissions including the access and operation rights of an object by an individual, a machine, and/or an application, and the circumstances under which the permission rights stand.

The techniques introduced here can be implemented by programmable circuitry (e.g., one or more microprocessors), software and/or firmware, special-purpose hardwired (i.e., non-programmable) circuitry, or a combination of such forms. Special-purpose circuitry can be in the form of, for example, one or more application-specific integrated circuits (ASICs), programmable logic devices (PLDs), field-programmable gate arrays (FPGAs), etc.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

Although the above Detailed Description describes certain embodiments and the best mode contemplated, no matter how detailed the above appears in text, the embodiments can be practiced in many ways. Details of the technology may vary considerably in its implementation details while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention not be limited by this Detailed Description, but rather by any claims that issue on an application based hereon. Accordingly, the disclosure of various embodiments is intended to be illustrative, but not limiting, of the scope of the technology.

1. A method comprising:
acquiring unscented digital content uploaded by an individual through a graphical user interface;
performing content analysis on individual frames of the unscented digital content to identify a frame that includes an identifiable feature;
proposing the frame as a candidate instance for a dispersal event;
receiving user input indicative of a confirmation of the candidate instance by the individual;
applying predictive analytics to identify a lead frame that leads the frame by a specified amount,
wherein the specified amount is variable based on a characteristic of a consumer who will be exposed to a scent produced by a stimuli delivery system; and
converting the unscented digital content into scented digital content by generating an instruction that prompts the stimuli delivery system to dispense a scented liquid to produce the scent responsive to a determination that a computing device has presented the lead frame.

2. The method of claim 1, wherein the characteristic is age, gender, smoking status, status of a health ailment known to affect sensory sensitivity, or any combination thereof.

3. The method of claim 2, wherein the lead time is automatically determined by a repurposing engine on behalf of an individual responsible for overseeing the creation of sensory stimuli events.

4. The method of claim 2, wherein the lead time is manually specified by an individual responsible for overseeing the creation of sensory stimuli events.

5. The method of claim 1, wherein said performing comprises:
performing image segmentation on the individual frames of the unscented digital content to identify digital representations of the identifiable feature.

6. The method of claim 1, wherein said performing comprises:
performing audio segmentation on the individual frames of the unscented digital content to identify audible descriptions of the identifiable feature.

7. The method of claim 1, wherein the lead time is based on a content type of the unscented digital content.

8. The method of claim 1, wherein the lead time is based on a duration of usage of the stimuli delivery system.

9. The method of claim 1, wherein the instruction is encoded in a video track, an audio track, or metadata of the scented digital content.

10. The method of claim 1, wherein the instruction is included in a distinct instruction set that accompanies the scented digital content and is separately executable by the stimuli delivery system.

11. A method comprising:
acquiring ordinary digital content;
performing content analysis on individual frames of the ordinary digital content to identify a frame that includes an identifiable feature;
proposing the frame as a candidate instance for a sensory stimulus event;
receiving user input indicative of a confirmation of the candidate instance;
applying predictive analytics to identify a lead frame that leads the frame by a specified amount; and
converting the ordinary digital content into sensory-enhanced digital content by generating a stimulus instruction that prompts a stimuli delivery system to perform an action responsive to a determination that a computing device has presented the lead frame,
wherein the action increases the realism of the identifiable feature included in the sensory-enhanced digital content by replacing real-world sensory stimuli with a controlled stimulus, and
wherein intensity of the controlled stimulus is based on a consumer characteristic, a usage characteristic, or any combination thereof.

12. The method of claim 11, wherein said acquiring comprises:
obtaining the ordinary digital content that is uploaded by an individual through a graphical user interface.

13. The method of claim 11, wherein said acquiring comprises:
retrieving the ordinary digital content from a network-accessible content database.

14. The method of claim 11, further comprising:
transmitting the stimulus instruction to the stimuli delivery system that is communicatively coupled to the computing device that presents the sensory-enhanced digital content to an individual.

15. The method of claim 14, wherein the computing device is a head-mounted device.

16. The method of claim 15, wherein the digital content includes virtual reality content or augmented reality content.

17. The method of claim 11, wherein the ordinary digital content includes visual content, and wherein the identifiable feature is a digital representation of an item, a character, a weather event, or a locale.

18. The method of claim 11, wherein the ordinary digital content only includes non-visual content, and wherein the identifiable feature is an audible description of an item, a character, a weather event, or a locale.

19. The method of claim 11, wherein the action includes performing at least one of;
dispensing a scented liquid to produce a scent;
modulating an air flow to simulate wind;
dispensing water to simulate rain; and
moderating a temperature of the scented liquid, the air flow, or the water.

20. A method comprising:
acquiring unscented visual content uploaded by an individual through a graphical user interface;
performing image segmentation on individual frames of the unscented visual content to identify a frame that includes a digital representation of an identifiable feature;
applying predictive analytics to identify a lead frame that leads the frame by a specified amount;
converting the unscented visual content into scented visual content by generating a first stimulus instruction that prompts a stimuli delivery system to dispense a scented liquid and produce a scent responsive to a determination that a computing device has presented the lead frame; and
configuring the lead time to increase proportional to duration of usage of the stimuli delivery system.

21. The method of claim 20, wherein the unscented visual content includes two-dimensional (2D) content or three-dimensional (3D) content.

22. The method of claim 20, wherein said configuring counteracts decreases in sensory sensitivity that occur after prolonged periods of use of the stimuli delivery system.

23. The method of claim 20, further comprising:
applying predictive analytics to identify a tail frame that follows the frame by a specified amount; and
generating a second stimulus instruction that prompts the stimuli delivery system to cease production of the scent.

24. The method of claim 20, further comprising:
generating a second stimulus instruction that prompts the stimuli delivery system to initiate a fan that controllably modulates an air flow to simulate wind.

25. The method of claim 24, further comprising:
generating a third stimulus instruction that prompts the stimuli delivery system to moderate a temperature of the air flow.

26. The method of claim 25, further comprising:
creating an instruction set that includes the first stimulus instruction, the second stimulus instruction, and the third stimulus instruction.

27. The method of claim 26, wherein the instruction set is encoded in a video track, an audio track, or metadata of the scented visual content.

28. The method of claim 26, wherein the instruction set accompanies the scented visual content and is separately executable by the stimuli delivery system.

29. The method of claim 20, wherein the specified amount is based on age, gender, or smoking status of a user.

30. The method of claim 20, wherein the identifiable feature is an item, a character, a weather event, or a locale.

31. A method for producing scents to increase the realism of a virtual reality environment traversed by an individual using a head-mounted display, the method comprising:
causing display of a virtual reality environment that includes multiple scented regions,
wherein each scented region of the multiple scented regions corresponds to a digital object associated a scent;
receiving input indicative of an instruction from an individual to move toward a desired location in the virtual reality environment;
establishing a current location of an individual within the virtual reality environment;
calculating a distance between the individual and each scented region of the multiple scented regions;
identifying a closest scented region to the current location of the individual,
wherein the closest scented region corresponds to a particular digital object associated with a particular scent;
determining that the distance between the closest scented region and the current location of the individual is less than a predetermined value; and
generating an instruction that controls an intensity of the particular scent to be produced by a scent delivery apparatus.

32. The method of claim 31, the multiple scented regions vary in size, shape, or any combination thereof.

33. The method of claim 31, wherein at least one scented region of the multiple scented regions is configured to move throughout the virtual reality environment over time.

34. The method of claim 31, wherein at least one scented region of the multiple scented regions is configured to disappear from the virtual reality environment following the expiration of a specified duration of time.

35. The method of claim 31, wherein said establishing, calculating, identifying, and determining are performed continually over time such that scents produced by the scent delivery apparatus can be varied in intensity based on proximity to each scented region of the multiple scented regions.
